(19) United States
(12) Patent Application Publication
(10) Pub. No.: US 2025/0216491 A1
(43) Pub. Date: Jul. 3, 2025

(54) METHODS, SYSTEMS, AND NON-TRANSITORY COMPUTER READABLE MEDIUMS FOR MAGNETIC RESONANCE IMAGING

(71) Applicant: WUHAN UNITED IMAGING LIFE SCIENCE INSTRUMENT CO., LTD., Wuhan (CN)

(72) Inventors: Zhongmiao WANG, Wuhan (CN); Renkuan ZHAI, Wuhan (CN); Xiaomao GONG, Shanghai (CN)

(73) Assignee: WUHAN UNITED IMAGING LIFE SCIENCE INSTRUMENT CO., LTD., Wuhan (CN)

(21) Appl. No.: 18/964,321

(22) Filed: Nov. 29, 2024

(30) Foreign Application Priority Data
Dec. 27, 2023 (CN) 202311844810.0

(51) Int. Cl. G01R 33/561; G01R 33/567

(52) U.S. Cl. CPC G01R 33/5616; G01R 33/5676

(57) ABSTRACT

Embodiments of the present disclosure provide a method, a system and a medium for magnetic resonance imaging (MRI), the method comprising: applying a gradient echo sequence to an object and performing a dummy scan on the object until a steady-state of the gradient echo sequence is reached; continuing the dummy scan and maintaining the steady-state of the gradient echo sequence; and in response to receiving a trigger signal while the gradient echo sequence is in the steady-state, acquiring a gradient echo magnetic resonance signal of the object. The system includes at least one processor; the at least one processor is configured to cause the system to perform the method for MRI.

### CROSS-REFERENCE

This application claims priority to Chinese application No. 202311844810.0 filed on Dec. 27, 2023, the entire contents of which are incorporated herein by reference.

### TECHNICAL FIELD

The present disclosure relates to a technical field of magnetic resonance imaging, and in particular, to methods, systems and non-transitory computer readable mediums for magnetic resonance imaging.

### BACKGROUND

Magnetic resonance imaging (MRI), which utilizes the phenomenon of magnetic resonance to image the object, has been a common medical imaging detection method. During the imaging process using magnetic resonance devices, the effects of factors such as involuntary movement and physiological activity of the object may result in motion artifacts in the image, which may affect the image-based diagnosis and research.

In gradient echo sequence imaging techniques, physiological triggering is often used to suppress motion artifacts. Achieving high-quality imaging may require acquiring gradient echo magnetic resonance signals while the sequence is in a steady state. However, traditional methods for maintaining this steady state often compromise imaging efficiency.

Therefore, there is a need for methods, systems, and non-transitory computer readable mediums for MRI that enhances imaging efficiency without sacrificing quality.

### SUMMARY

One or more embodiments of the present disclosure provide a method for MRI. the method includes: applying a gradient echo sequence to an object and performing a dummy scan on the object until a steady-state of the gradient echo sequence is reached; continuing the dummy scan and maintaining the steady-state of the gradient echo sequence; and in response to receiving a trigger signal while the gradient echo sequence is in the steady-state, acquiring a gradient echo magnetic resonance signal of the object.

One or more embodiments of the present provide a system for MRI. The system includes at least one processor. The at least one processor is configured to apply a gradient echo sequence to an object and perform a dummy scan on the object until a steady-state of the gradient echo sequence is reached; continue the dummy scan and maintain the steady-state of the gradient echo sequence; in response to receiving a trigger signal while the gradient echo sequence is in the steady-state, acquire a gradient echo magnetic resonance signal of the object.

One or more embodiments of the present disclosure provide a non-transitory computer readable medium. The medium stores instructions, the instructions, when executed by at least one processor, causing the at least one processor to implement a method for MRI. The method for MRI includes: applying a gradient echo sequence to an object and performing a dummy scan on the object until a steady-state of the gradient echo sequence is reached; continuing the dummy scan and maintaining the steady-state of the gradient echo sequence; and in response to receiving a trigger signal while the gradient echo sequence is in the steady-state, acquiring a gradient echo magnetic resonance signal of the object.

The above-described method, system and medium for MRI performs the dummy scan on the object before a physiological trigger signal is detected, and performs the acquisition of the gradient echo magnetic resonance signal after the gradient echo sequence is in the steady-state. This not only enables the acquisition of the gradient echo magnetic resonance signal of the object while the gradient echo sequence is in the steady-state, improving the quality of the imaging, but also eliminates the need to perform a sufficient number of dummy scans from the beginning each time the physiological trigger signal is detected to achieve the steady-state of the gradient echo sequence. The time before the steady-state acquisition may be greatly saved, thus improving the acquisition efficiency of the gradient echo magnetic resonance signal and greatly saving the magnetic resonance imaging time.

### BRIEF DESCRIPTION OF THE DRAWINGS

The present disclosure may be further described in terms of exemplary embodiments, which may be described in detail with reference to the drawings. These embodiments are not limiting, and in these embodiments, the same reference numerals in the various drawings represent similar structures, and where:

FIG. 1 is a sequence diagram of a traditional gradient echo sequence combined with a physiological triggering technique during an MRI process according to some embodiments of the present disclosure;

FIG. 2 is an application scenario diagram of an MRI system according to some embodiments of the present disclosure.

FIG. 3 is an exemplary structural diagram of a computer device according to some embodiments of the present disclosure;

FIG. 4 is an exemplary structural diagram of an MRI device according to some embodiments of the present disclosure;

FIG. 5 is a flowchart of an exemplary MRI process according to some embodiments of the present disclosure;

FIG. 6 is an exemplary schematic diagram of a scanning feature prediction model according to some embodiments of the present disclosure;

FIG. 7 is an exemplary flowchart for determining a triggering time point of a next trigger signal according to some embodiments of the present disclosure;

FIG. 8 is an exemplary flowchart for performing a dummy scan on an object according to some embodiments of the present disclosure;

FIG. 9 is an exemplary flowchart for acquiring a gradient echo magnetic resonance signal of an object according to some embodiments of the present disclosure;

FIG. 10 is a sequence diagram during an MRI process according to some embodiments of the present disclosure;

FIG. 11 is another sequence diagram during an MRI process according to some embodiments of the present disclosure;

FIG. 12 is another sequence diagram during an MRI process according to some embodiments of the present disclosure;

FIG. 13 is another sequence diagram during an MRI process according to some embodiments of the present disclosure;

FIG. 14 is another sequence diagram during an MRI process according to some embodiments of the present disclosure;

FIG. 15A is a magnitude image corresponding to a magnetic resonance image according to some embodiments of the present disclosure; and

FIG. 15B is a phase image corresponding to a magnetic resonance image according to some embodiments of the present disclosure.

### DETAILED DESCRIPTION

In order to more clearly illustrate the technical solutions of the embodiments of the present disclosure, the accompanying drawings required to be used in the description of the embodiments will be briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. The present disclosure can be applied to other similar scenarios based on these drawings without creative labor. Unless obviously obtained from the context or the context illustrates otherwise, the same numeral in the drawings refers to the same structure or operation.

It should be understood that the terms “system,” “device,” as used herein, “unit,” and/or “module” as used herein is a way to distinguish between different components, elements, parts, sections or assemblies at different levels. However, said words may be replaced by other expressions if other words accomplish the same purpose.

As shown in the disclosure and claims, unless the context clearly indicates an exception, words such as “one,” “a,” “an,” and/or “the” do not specifically refer to the singular, but may also include the plural. Generally, the terms “including,” and “comprising” suggest only the inclusion of clearly identified steps and elements. In general, the terms “including,” and “comprising” only suggest the inclusion of explicitly identified steps and elements that do not constitute an exclusive list, and the method or device may also include other steps or elements.

Flowcharts are used in this disclosure to illustrate operations performed by a system according to embodiments of this disclosure. It should be appreciated that the preceding or following operations are not necessarily performed in an exact sequence.

Instead, steps can be processed in reverse order or simultaneously. Also, it is possible to add other operations to these processes or remove a step or steps from them.

In the field of imaging technology, Magnetic resonance imaging (MRI), which utilizes the phenomenon of magnetic resonance to image an object, has been a common medical imaging detection method. During the imaging process using a magnetic resonance device, the effects of factors such as involuntary movement and physiological activity of the object may result in motion artifacts in the image, which may affect the image-based diagnosis and research.

Traditional Gradient Recalled Echo (GRE) sequence techniques can be combined with physiological triggering techniques to suppress motion artifacts. One combination is to perform unsteady-state GRE K-space data acquisition directly after a physiological trigger point, and the other combination is to set a reasonable number of dummy scans before the physiological trigger point, so that the GRE signal reaches the steady-state before the K-space data acquisition.

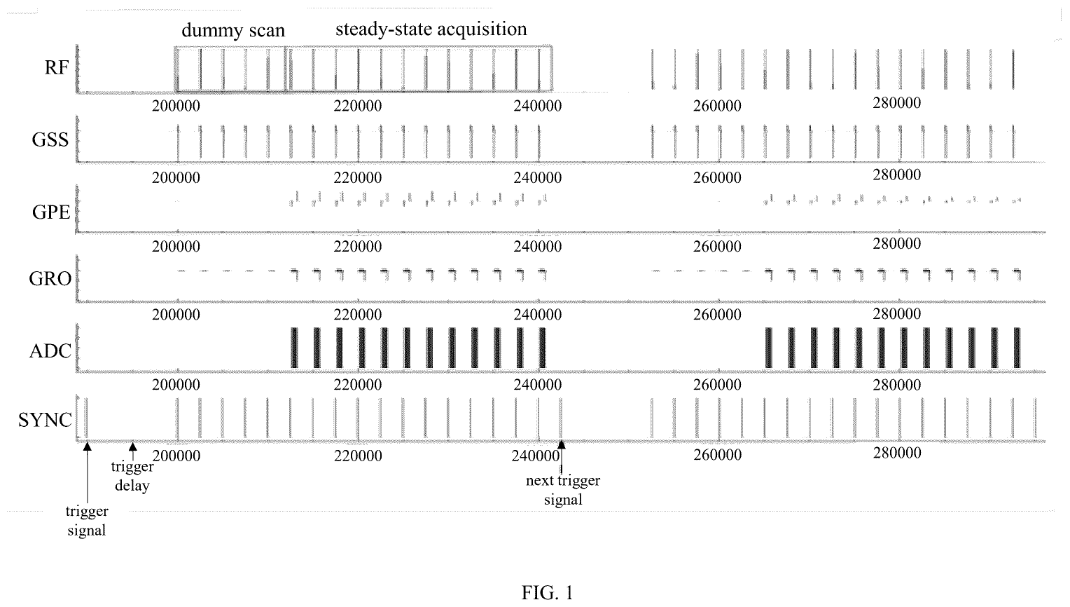

FIG. 1 is a sequence diagram of a traditional gradient echo sequence combined with a physiological triggering technique during an MRI process according to some embodiments of the present disclosure. As shown in FIG. 1, after a physiological trigger point and a trigger delay phase, a certain number of dummy scans are performed to make the GRE signal reach a steady-state. Data is acquired when the GRE signal is in the steady-state (e.g., a steady-state acquisition) until a next physiological trigger point arrives. Repeating the above process to perform an MRI process. In FIG. 1, RF represents radio frequency pulse. GRO represents a readout axis (e.g., a frequency coding axis), and a value of GRO represents a gradient field intensity along the frequency coding axis. GPE represents a phase encoding axis, and a value of GPE represents a gradient field intensity along the phase encoding axis. GSS represents a slice selection axis, and a value of GSS represents a gradient field intensity along the slice selection axis. ADC represents a signal acquisition axis and a SYNC represents a physiological trigger signal axis.

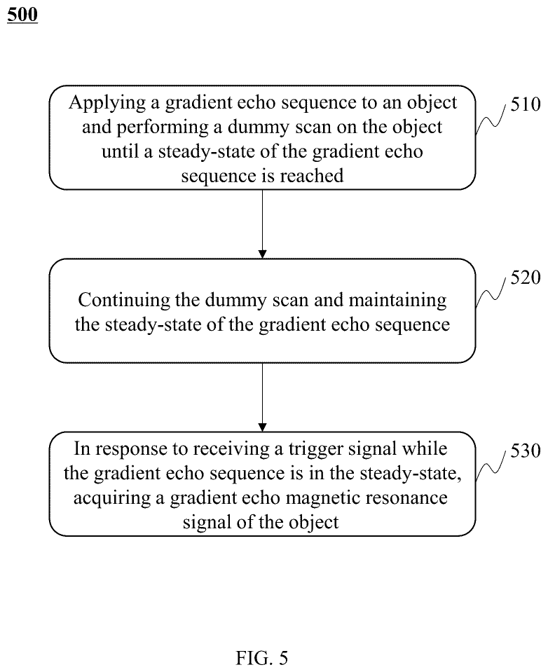

If the unsteady-state GRE K-space data acquisition is performed after the physiological trigger point, it will result in poor imaging quality and a small range of applications due to the existence of inconsistencies in K-space data. For the scheme of performing the dummy scans after the physiological trigger point to reach the steady-state of the GRE, since the number of dummy scans to reach the steady-state is often several dozens, and the time of each dummy scan is a repetition time (TR) of GRE sequences, this will occupy a long period of time and reduce the efficiency of data acquisition. And the number of dummy scans is also related to sequence parameters, and the number of dummy scans will be larger under some sequence parameters, which will lead to a more inefficient acquisition of the data, in turn reduce the imaging efficiency. In this regard, the present disclosure provides a method for MRI, as particularly described in FIG. 5-FIG. 9 and the related descriptions thereof.

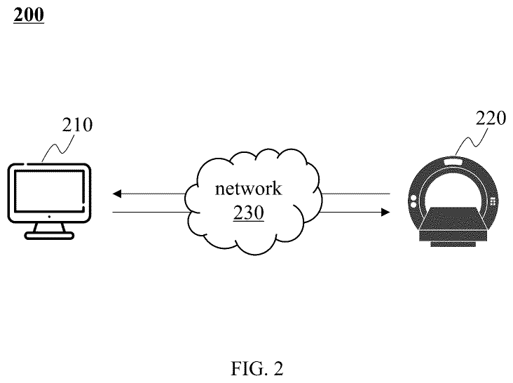

FIG. 2 is an application scenario diagram of an MRI system according to some embodiments of the present disclosure. The MRI system provided in embodiments of the present disclosure may be applied in an application scenario as shown in FIG. 2.

As shown in FIG. 2, the application scenario 200 includes a computer device 210 and a magnetic resonance device 220. In some embodiments, the computer device 210 communicates with the magnetic resonance device 220 via a network 230. In some embodiments, the computer device 210 is a terminal device of a user. The magnetic resonance device 220 is configured to communicate with the terminal device. For example, in response to receiving configuration information from the user via the terminal device, the magnetic resonance device 220 performs operations based on the configuration information. The configuration information is configured to cause the magnetic resonance device 220 to perform the operations. The operations includes a method for MRI described in some embodiments of the disclosure.

The magnetic resonance device 220 is configured to scan an object, obtain gradient echo magnetic resonance signals, and transmit the gradient echo magnetic resonance signal to the computer device 210. More detailed descriptions of the object can be found in FIG. 5 and its associated descriptions.

In some embodiments, the magnetic resonance device 220 acquires a large amount of unprocessed raw data by performing a magnetic resonance scan on the object. In some embodiments, the magnetic resonance device 220 performs the magnetic resonance scan on the object in a certain scanning sequence. The scanning sequence includes, but is not limited to, a free induction decay (FID) sequence, a self-selected echo (SE) sequence, a gradient echo sequence (GRE), a heteroscedastic sequence (HS), or the like. In some embodiments, the magnetic resonance device 220 performs the magnetic resonance scan on the object using different scanning sequences for different objects. For example, the magnetic resonance device 220 employs an Ax SE T1 scanning sequence on a conventional head and a Cor SE T1 scanning sequence on a pituitary gland. In some embodiments, the magnetic resonance device 220 includes a 1.5T magnetic resonance device, a 3T magnetic resonance device, a 5T magnetic resonance device, a 7T magnetic resonance device, or the like.

In some embodiments, the magnetic resonance device 220 sends the gradient echo magnetic resonance signals through the network 230 to the computer device 210 for processing. In some embodiments, the magnetic resonance device 220 detects relevant data or instructions from the computer device 210 to perform the magnetic resonance scan.

The foregoing description of the magnetic resonance device 220 is for illustrative purposes only and is not intended to limit the scope of this disclosure.

In some embodiments, the computer device 210 is configured to reconstruct a magnetic resonance image of the object based on the gradient echo magnetic resonance signals. The computer device 210 includes, but is not limited to, an industrial computer, a laptop, a tablet, an embedded device, or the like.

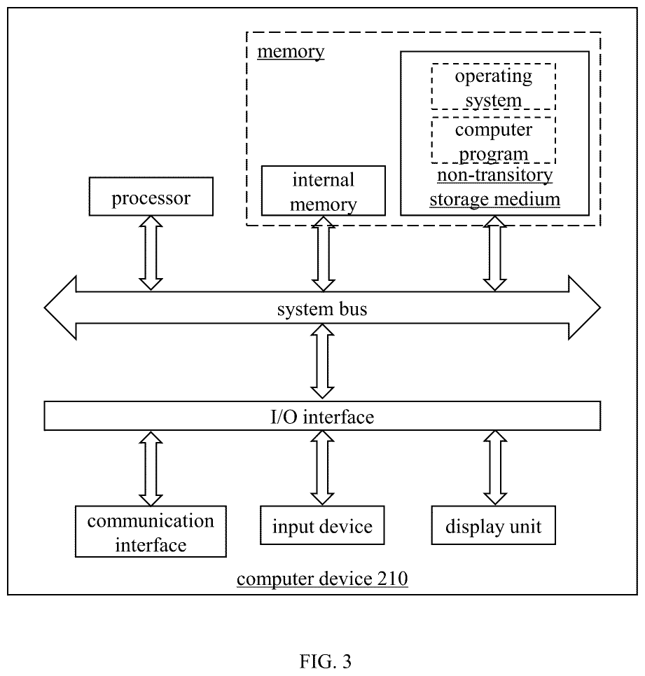

FIG. 3 is an exemplary structural diagram of a computer device according to some embodiments of the present disclosure. As shown in FIG. 3, the computer device 210 includes a processor, a memory, a communication interface, an input device, and a display unit connected via a system bus.

The processor is configured to provide computing and control capabilities. The processor can process data and/or information obtained from the magnetic resonance device 220, the memory, and/or the input device. For example, the processor reconstructs a magnetic resonance image of an object based on gradient echo magnetic resonance signals sent by the magnetic resonance device 220.

The memory can store data, instructions, and/or any other information. For example, the memory stores raw data files acquired by the magnetic resonance device 220. For another example, the memory stores data acquired from the magnetic resonance device 220, the processor, and/or the input device. In some embodiments, the memory includes an internal memory and a non-transitory storage medium. The non-transitory storage medium stores an operating system and a computer program, and the internal memory provides an environment for the operation of the operating system and the computer program in the non-transitory storage medium. The computer program is executed by the processor to implement a method for MRI described in some embodiments of the disclosure.

The communication interface is configured for wired or wireless communication with external terminals. The wireless communication may be realized via WIFI, mobile cellular networks, NFC, or other technologies.

The input device and the display unit are configured to realize the interaction between the user and the computer device. For example, the display unit includes an LCD display, an e-ink display, or the like. The input unit may be a touch layer overlaying the display, a keypad, a trackball, or a touchpad provided on a housing of the computer device, an external keyboard, a touchpad, or a mouse, or the like.

It should be noted that the foregoing description is provided for illustrative purposes only and is not intended to limit the scope of the present disclosure. For a person of ordinary skill in the art, a wide variety of variations and modifications may be made under the guidance of the contents of this disclosure. Features, structures, methods, and other characteristics of the exemplary embodiments described herein may be combined in various ways to obtain additional and/or alternative exemplary embodiments.

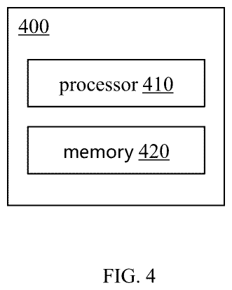

FIG. 4 is an exemplary structural diagram of an MRI device according to some embodiments of the present disclosure. The method for MRI provided in embodiments of the present disclosure may be realized by an MRI device as shown in FIG. 4.

As shown in FIG. 4, the MRI device 400 includes at least one processor 410 and at least one memory 420.

The at least one memory 420 is configured for storing computer instructions, and the at least one processor 410 is for executing at least a portion of the computer instructions to implement the method for MRI.

In some embodiments, the at least one processor 410 is configured to perform a dummy scan on an object, apply a gradient echo sequence to the object during a dummy scan phase until a steady-state of the gradient echo sequence is reached, and continue the dummy scan and maintain the steady-state of the gradient echo sequence. The at least one processor 410 is configured to, in response to receiving a trigger signal while the gradient echo sequence is in the steady-state, acquire a gradient echo magnetic resonance signal of the object. The at least one processor 410 is configured to reconstruct, based on the gradient echo magnetic resonance signal, a magnetic resonance image of the object.

In some embodiments, the at least one processor 410 is further configured to, in response to receiving the trigger signal, perform the dummy scan on the object during a trigger delay phase corresponding to the trigger signal. During the steady-state acquisition phase after the trigger delay phase ends, the at least one processor 410 is configured to acquire the gradient echo magnetic resonance signal of the object.

In some embodiments, the at least one processor 410 is further configured to perform a next dummy scan on the object until a next trigger signal is received.

In some embodiments, the at least one processor 410 is further configured to apply a first radio frequency pulse to the object and simultaneously apply a first selective slice gradient field to the object without acquiring signals.

In some embodiments, the at least one processor 410 is also configured to determine, based on physiological movement features of the object during the trigger delay phase, a number of data acquisitions within the steady-state acquisition phase.

In some embodiments, the at least one processor 410 is further configured to, in response to determining that a scanning feature of a current phase satisfies a first preset condition, trigger a stop instruction.

In some embodiments, the at least one processor 410 is further configured to determine, based on the scanning feature of the current phase, a scanning feature of a next phase. In response to determining that the scanning feature of the next phase satisfies the first preset condition, the at least one processor 410 is configured to trigger the stop instruction.

In some embodiments, the at least one processor 410 is further configured to determine, by processing the scanning feature of the current phase based on a scanning feature prediction model, the scanning feature of the next phase.

In some embodiments, the at least one processor 410 is further configured to, in response to determining that a scanning feature of a last phase satisfies the first preset condition and a stopping time duration satisfies a second preset condition, trigger a start scanning instruction.

In some embodiments, the at least one processor 410 is further used configured to perform a next dummy scan of the object after a first preset length of time from a time point that acquiring the gradient echo magnetic resonance signal, until receiving a next trigger signal.

In some embodiments, the at least one processor 410 is further configured to determine the first preset length of time based on a triggering time point of the next trigger signal, a duration of a trigger delay phase corresponding to the trigger signal, and a steady-state recovery time.

In some embodiments, the at least one processor 410 is further configured to determine, based on current trigger-signal information, physiological movement features of the object before an end of the steady-state acquisition phase, and object information, a time interval from a triggering time point of a current trigger signal to the triggering time point of the next trigger signal. Based on the time interval and the triggering time point of the current trigger signal, the at least one processor 410 is configured to determine the triggering time point of the next trigger signal.

In some embodiments, the at least one processor 410 is further configured to apply a first dephasing gradient field to the object after applying the first selective slice gradient field to the object.

In some embodiments, the at least one processor 410 is further used configured to apply a second radio frequency pulse to the object and simultaneously apply a second selective slice gradient field to the object. After a second preset length of time, the at least one processor 410 is configured to apply a phase-encoded gradient field and a frequency-encoded gradient field to the object to acquire the gradient echo magnetic resonance signal of the object.

In some embodiments, the at least one processor 410 is also configured to apply a second dephasing gradient field to the object before applying the frequency-encoded gradient field to the object. After applying the frequency-encoded gradient field to the object, the at least one processor 410 is configured to apply a third dephasing gradient field to the object.

FIG. 5 is an exemplary flowchart of a method for MRI according to some embodiments of the present disclosure. In some embodiments, a process 500 is executed by a computer device. For example, the process 500 is stored in memory in the form of a program or instruction, and the processor implements the process 500 when it executes the program or instruction. The schematic diagram of the operation of process 500 presented below is illustrative. In some embodiments, the processor completes the process utilizing one or more additional operations not described and/or one or more operations not discussed. Additionally, the order of the operations of process 500 illustrated in FIG. 5 and described below is not limiting.

Step 510, a gradient echo sequence is applied to an object and a dummy scan is performed on the object until a steady-state of the gradient echo sequence is reached.

In some embodiments, the object may be biological or non-biological. For example, the object includes a patient, an artificial object, or the like. In some embodiments, the object includes a particular portion of the body, such as, a head, a neck, a chest, or the like, or any combination thereof. In some embodiments, the object includes a particular organ, such as, a liver, a kidney, a pancreas, a bladder, a uterus, a rectum, or the like, or any combination thereof. In some embodiments, the object includes a region of interest (ROI), such as, a tumor, a nodule, or the like.

The dummy scan is an operation in which only radio frequency pulses are emitted without acquiring a magnetic resonance signal.

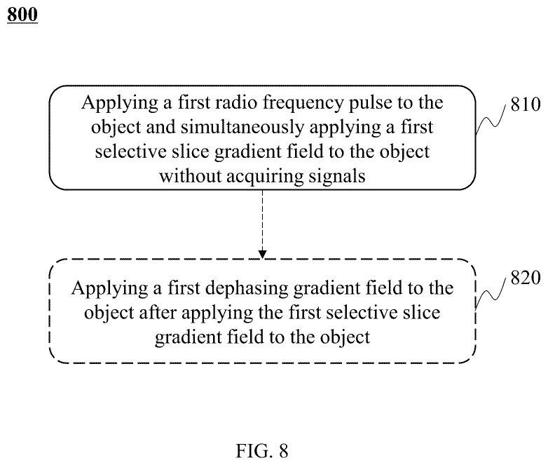

When an MRI process is required, the computer device sends a control signal to an MRI device to allow the MRI device to perform the dummy scan on the object. That is, after the MRI device is turned on and before the magnetic resonance signal is acquired, the object may be performed dummy scan (which may also be referred to as pre dummy scan) for a certain period of time or a certain number of times. During the dummy scan (or the pre dummy scan), the magnetic resonance signal is not acquired. The purpose of the pre dummy scan after the MRI device is turned on is to make the gradient echo sequence reach the steady-state. See FIG. 8 for more descriptions of the dummy scan.

In some embodiments, the computer device may send the control signal to the MRI device to apply the gradient echo sequence to the object and perform the dummy scan on the object.

The gradient echo sequence reaching the steady-state refers that the gradient echo no longer changes significantly over time and reaches dynamic equilibrium. For example, the gradient echo sequence may be considered to reach the steady-state when magnitudes of the gradient echoes no longer change significantly (e.g., a change of the magnitudes of the gradient echoes is less than a certain threshold).

The gradient echo sequence reaching the steady-state may include both a longitudinal magnetization vector (a magnetization vector in a longitudinal direction) and a transverse magnetization vector (a magnetization vector in a transverse direction) of the gradient echo sequence reach the steady-state. The longitudinal magnetization vector reaching the steady-state refers that after a plurality of excitations, the magnetization vector in the longitudinal direction remains in a relatively stable state. The transverse magnetization vector reaching the steady-state refers that after a plurality of excitations, the magnetization vector in the transverse direction remains in a relatively stable state.

During a dummy scan phase, the computer device detects, in real time, whether the gradient echo sequence reaches the steady-state. For example, the computer device monitors, in real time, the change in the longitudinal magnetization vector and the change in the transverse magnetization vector. Based on the change in the longitudinal magnetization vector and the change in the transverse magnetization vector, the computer device then determines whether the gradient echo sequence has reached the steady-state or not.

Step 520, the dummy scan is continued and the steady-state of the gradient echo sequence is maintained.

In some embodiments, upon detecting that the gradient echo sequence is in the steady-state, the computer device may control the MRI device to continuously perform the dummy scan on the object to maintain the steady-state of the gradient echo sequence.

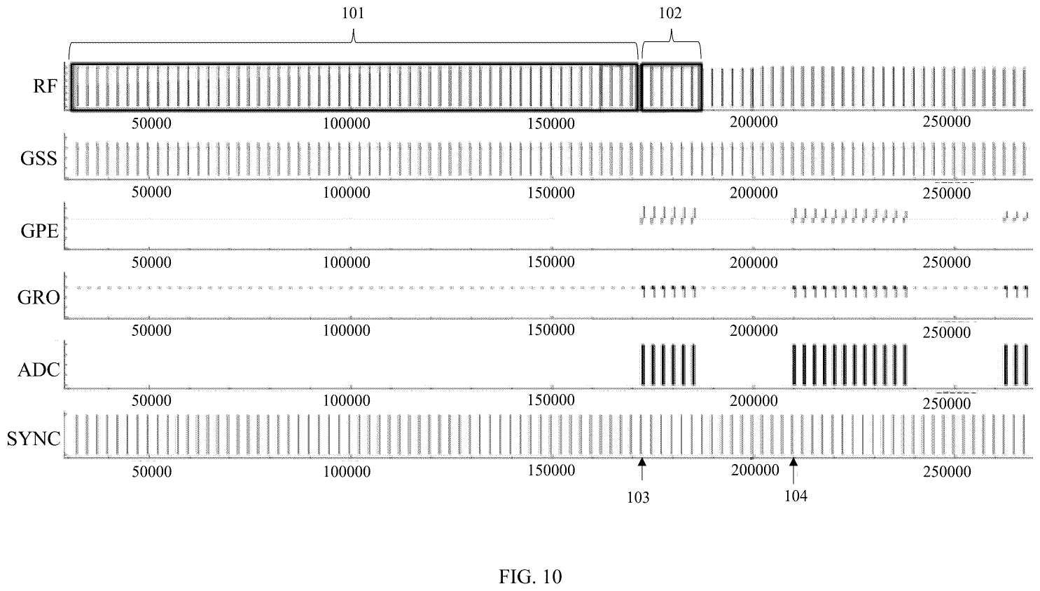

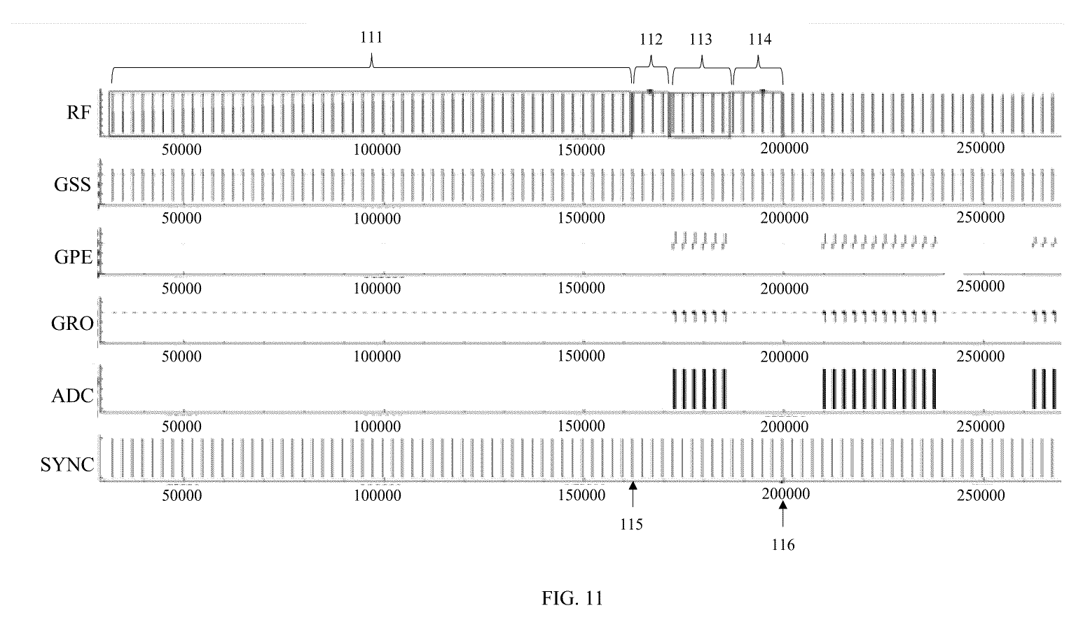

In some embodiments, the dummy scan may be continued until a trigger signal is detected. That is, the dummy scan ends when the trigger signal is detected. In other embodiments, the dummy scan may be continued until after the trigger signal is detected. That is, the dummy scan may be continued during the trigger delay phase after the trigger signal is detected until the trigger delay phase ends. See FIG. 10 and FIG. 11 for more descriptions.

Step 530, in response to receiving a trigger signal while the gradient echo sequence is in the steady-state, a gradient echo magnetic resonance signal of the object is acquired.

The trigger signal is the control signal that controls the MRI device to start an acquisition of the gradient echo magnetic resonance signal. When the computer device detects the trigger signal, a signal acquisition phase is reached, and the MRI device may acquire the gradient echo magnetic resonance signal of the object.

In some embodiments, the trigger signal includes at least one of a physiological trigger signal, a temporal trigger signal, a motion trigger signal, or the like. The physiological trigger signal is associated with physiological signals of the object. In some embodiments, the physiological signals of the object include, signals of physiological movements such as real or simulated heartbeats, breathing, or the like.

In some embodiments, the physiological trigger signal includes a signal that is triggered and generated at a particular phase point of a physiological exercise cycle of the object. The particular phase point of the physiological exercise cycle may include an end position of a rising edge of a respiratory signal, a R-wave position of an ECG signal, or the like.

In some embodiments, when the gradient echo sequence is in the steady-state, the computer device monitors the physiological signals of the object and determines that the physiological trigger signal is detected upon a detection of the particular phase point of the physiological motion cycle (e.g., a detection of the particular phase point such as the end position of the rising edge of the respiratory signal and/or a detection of the R-wave position of the ECG signal).

The temporal trigger signal is a time-dependent trigger signal. In some embodiments, the temporal trigger signal includes a signal that is triggered and generated at a point in time at a preset time interval after the gradient echo sequence reaches the steady-state. The preset time interval may be a system default value, an empirical value, a human pre-set value, or the like, or any combination thereof, and the preset time interval may be determined according to actual needs.

In some embodiments, under a circumstance that the gradient echo sequence is in the steady-state, the computer device may determine that the temporal trigger signal is detected at a point in time after the preset time interval after the gradient echo sequence reaches the steady-state.

The motion trigger signal is associated with motion sensing signals of the object. The motion sensing signals are signals that reflect motion characteristics of the object. The motion characteristics may include position information, motion amplitude, motion frequency, or the like of the object. The motion amplitude may be a position offset distance of the object during two position detections. The motion frequency may be the number of times the object moves over a period of time. The motion amplitude and the motion frequency may be determined based on the position information of the object over a period of time. The position information of the object may be obtained by any feasible sensing device such as a motion sensor, an infrared sensor, or the like.

In some embodiments, the motion trigger signal includes a signal the is triggered and generated when the motion amplitude of the object is less than a preset amplitude threshold. The preset amplitude threshold is a threshold condition associated with the motion amplitude of the object. The preset amplitude threshold may be a system default value, an empirical value, a human pre-set value, or the like, or any combination thereof, and may be determined according to actual needs.

In some embodiments, the computer device determines the motion amplitude of the object based on a variation of the position information of the object in a plurality of motion sensing signals.

When the motion amplitude of the object is too large, the acquired gradient echo magnetic resonance signal contains a plurality of interfering signals, and a reconstructed magnetic resonance image is of poor quality. In order to improve the quality of the magnetic resonance image, it is necessary to generate the motion trigger signal when the motion amplitude of the object is less than the preset amplitude threshold. In some embodiments, the preset amplitude threshold is positively correlated with a size of the object.

In some embodiments, when the gradient echo sequence is in the steady-state, the computer device monitors the motion sensing signals of the object and determines that the motion trigger signal is detected when the motion amplitude of the object is detected to be less than the preset amplitude threshold.

The gradient echo magnetic resonance signal is an analog signal with spatially encoded information.

In some embodiments, the computer device, in response to detecting the trigger signal, sends the control signal to the MRI device to cause the MRI device to acquire the gradient echo magnetic resonance signal of the object. That is, in this embodiment, the detection of the trigger signal indicates a steady-state acquisition phase is entered.

The steady-state acquisition phase is a phase in which the gradient echo magnetic resonance signal is acquired when the gradient echo sequence is in the steady-state. In some embodiments, a duration of the steady-state acquisition phase is preset by the user based on actual experience.

In some embodiments, the computer device, in response to detecting the trigger signal, determines that the steady-state acquisition phase is entered and sends the control signal to the MRI device to cause the MRI device to acquire the gradient echo magnetic resonance signal of the object.

In an optional embodiment, a sequence diagram based on a combination of the gradient echo sequence and the trigger signal is provided as shown in FIG. 10.

FIG. 10 is a sequence diagram during an MRI process according to some embodiments of the present disclosure, and FIG. 10 corresponds to a sequence diagram in which the gradient echo magnetic resonance signal is directly acquired after the trigger signal is detected. As shown in FIG. 10, 101 denotes a dummy scan phase, 102 denotes a steady-state acquisition phase, 103 denotes a current trigger signal, and 104 denotes a next trigger signal. The MRI device is in the dummy scan phase 101 until the current trigger signal 103 is detected, and the object is continuously dummy scanned during the dummy scan phase 101. When the current trigger signal 103 is detected, the MRI device enters the steady-state acquisition phase 102, and the gradient echo magnetic resonance signal of the object is acquired in the steady-state acquisition phase 102. More descriptions of symbols shown in FIG. 10, such as RF, GRO, GPE, GSS, ADC, and SYNC are described in FIG. 1 and its related descriptions.

In other embodiments, the computer device, in response to receiving the trigger signal, performs the dummy scan on the object during a trigger delay phase corresponding to the trigger signal. The computer device, during the steady-state acquisition phase after the trigger delay phase ends, acquires the gradient echo magnetic resonance signal of the object.

After the trigger signal is generated, a corresponding trigger delay phase exists. The trigger delay phase is a time period between a point in time when the trigger signal is detected and a point in time when the acquisition of the gradient echo magnetic resonance signal begins. For example, the trigger delay phase lasts for 1s, 10s, or the like.

In some embodiments, a duration of the trigger delay phase is preset by the user based on actual experience.

In some embodiments, in response that the computer device detects the trigger signal, the MRI device continues to perform the dummy scan (which may also be referred to as a delay dummy scan) on the object within the trigger delay phase. In this embodiment, the delay dummy scan maintains the steady-state of the gradient echo sequence and the acquisition of the gradient echo magnetic resonance signal is not performed during the trigger delay phase.

In some embodiments, the computer device, in response to detecting the trigger signal, applies the gradient echo sequence to the object and performs the dummy scan on the object during the trigger delay phase corresponding to the trigger signal.

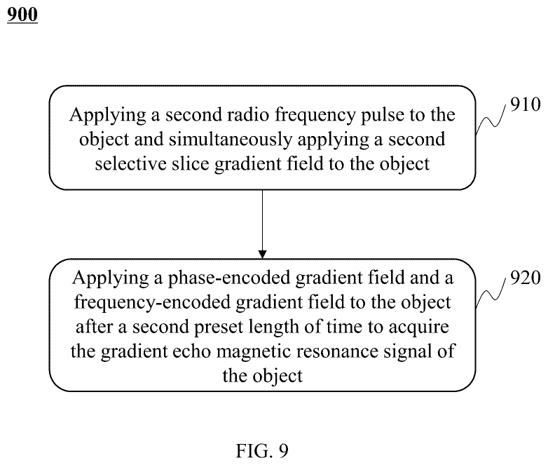

It should be noted that the gradient echo sequence applied to the object after the trigger signal is detected may be different from the gradient echo sequence applied to the object during the dummy scan phase. For example, the gradient echo sequence applied to the object after the trigger signal is detected is related to data acquisition, and the gradient echo sequence applied to the object during the dummy scan phase is related to radio frequency excitation. For more descriptions on the gradient echo sequence applied to the object during the dummy scan phase, see FIG. 8 and its related instructions. For more on the gradient echo sequence applied to the object after the trigger signal is detected, see FIG. 9 and its related instructions.

In some embodiments, during the steady-state acquisition phase after the trigger delay phase ends, the computer device sends the control signal to the MRI device to cause the MRI device to acquire the gradient echo magnetic resonance signal of the object. That is, in this embodiment, the end of the trigger delay phase indicates the steady-state acquisition phase is entered. The trigger delay phase may end when the duration of the delay dummy scan equals the duration of the trigger delay phase.

As shown in FIG. 11, 111 denotes the dummy scan phase, 112 denotes the trigger delay phase, and 113 denotes the steady-state acquisition phase. The MRI device is in the dummy scan phase 111 before detecting the current trigger signal 115, and the dummy scan of the object continues during the dummy scan phase 111. After detecting the current trigger signal 115, the MRI device is in the trigger delay phase 112, and the MRI device continues to perform the delay dummy scan in the trigger delay phase 112, during which the gradient echo magnetic resonance signal is not acquired. After the end of the trigger delay phase 112, the steady-state acquisition phase 113 is entered, and the MRI device acquires the gradient echo magnetic resonance signal of the object in the steady-state acquisition phase 113. More descriptions of symbols shown in FIG. 10, such as RF, GRO, GPE, GSS, ADC, and SYNC are described in FIG. 1 and its related descriptions.

In some embodiments, during the steady-state acquisition phase after the trigger delay phase ends, the computer device, based on a number of data acquisitions during the steady-state acquisition phase, sends the control signal to the MRI device to control the MRI device to acquire the gradient echo magnetic resonance signal of the object for the number of data acquisitions. In this embodiment, the number of data acquisitions in the steady-state acquisition phase may be preset by the user based on actual experience.

In some embodiments, the computer device controls the MRI device to acquire the gradient echo magnetic resonance signal during the steady-state acquisition phase in accordance with the number of data acquisitions during the steady-state acquisition phase. The number of data acquisitions within the steady-state acquisition phase may be a system default value, an empirical value, an artificially preset value, or the like, or any combination thereof, and may be set according to the actual needs.

In some embodiments, the computer device determines, based on physiological movement features of the object during the trigger delay phase, the number of data acquisitions within the steady-state acquisition phase.

The physiological movement features are associated with the physiological motion of the object. For example, the physiological movement features include a respiratory cycle, a heartbeat cycle, or the like of the object. The computer device may determine the physiological movement features based on the physiological signals of the object. For example, the computer device determines the respiratory cycle and the heartbeat cycle of the object based on respiratory signals and heartbeat signals of the object.

In some embodiments, the computer device constructs a reference vector library based on historical data. The reference vector library includes a plurality of reference vectors. Each of the plurality of reference vectors is a feature vector corresponding to historical physiological movement features during a trigger delay phase of a historical object corresponding to a magnetic resonance image that satisfies a requirement in the historical data. The reference vector library also includes an actual number of data acquisitions in the steady-state acquisition phase for the historical object corresponding to each reference vector. The computer device constructs a to-be-matched vector based on the physiological movement features of the object during the trigger delay phase, matches the to-be-matched vector in the reference vector library, and identifies a reference vector having a smallest vector distance from the to-be-matched vector. Actual number of data acquisitions corresponding to the identified reference vector is determined as the number of data acquisitions of the object in the steady-state acquisition phase. The magnetic resonance image satisfying the requirement may be that an image quality of the magnetic resonance image reaches a preset quality threshold. The vector distance includes, but are not limited to, a Euclidean distance, a cosine distance, or the like.

The closer the magnetic resonance image is to the actual condition of the object, the higher the image quality of the magnetic resonance image. The image quality and the preset quality threshold for the magnetic resonance image may be determined by relevant technicians (e.g., physicians, nurses, or the like) based on historical experience.

As shown in FIG. 11, the number of data acquisitions within the steady-state acquisition phase 113 labeled in FIG. 11 is 6 (the number of ADC pulses for the steady-state acquisition phase in FIG. 11 is 6). When the number of data acquisitions equals 6, the steady-state acquisition phase 113 ends and the MRI device no longer acquires the gradient echo magnetic resonance signal.

The image quality of the magnetic resonance image is positively correlated with the number of gradient echo magnetic resonance signals. The less number of data acquisitions is, the shorter the data acquisition time is, and the fewer motion artifacts of the magnetic resonance image includes, however the longer the scan time is. Some embodiments of the present disclosure, by determining the number of data acquisitions in the steady-state acquisition phase, the steady-state acquisition phase is controlled in advance, which is conducive to achieving an optimal acquisition effect and obtaining a high quality magnetic resonance image.

In some embodiments of the present disclosure, the steady-state of the gradient echo sequence is maintained by performing the dummy scan on the object during the trigger delay phase after receiving the trigger signal. The gradient echo sequence is in the steady-state when the gradient echo magnetic resonance signal is acquired after the trigger delay phase, which results in a good quality of the acquired gradient echo magnetic resonance signal, and in turn improves the quality of the magnetic resonance image.

For more on acquiring the gradient echo magnetic resonance signal of the object see FIG. 9 and its associated description.

In some embodiments, at the end of a steady-state acquisition phase, the computer device sends the control signal to the MRI device to cause the MRI device to stop acquiring until the next steady-state acquisition phase is performed when the next trigger signal is detected. In some embodiments, at the end of a steady-state acquisition phase, before the next trigger signal is detected, the computer device sends the control signal to the MRI device to cause the MRI device to perform a next dummy scan either directly on the object, or to perform the next dummy scan on the object after a period of time. See related content after step 530 for more description.

In some embodiments, the computer device is further configured to reconstruct a magnetic resonance image of the object based on the gradient echo magnetic resonance signal.

The MRI device may transmit the gradient echo magnetic resonance signal of the object to the computer device after completing a data acquisition process. The gradient echo magnetic resonance signal obtained by the MRI device is an electrical signal (an analog signal) with spatially encoded information. After the gradient echo magnetic resonance signal is detected by the computer device, the computer device may perform analog-to-digital conversion of the gradient echo magnetic resonance signal to a digital signal. The computer device fills the digital signal into the K-space in accordance with a preset filling algorithm to obtain a corresponding K-space dataset. The computer device performs an image reconstruction based on the K-space dataset to obtain the magnetic resonance image of the object.

In some embodiments of the present disclosure, the dummy scan is performed on the object before the trigger signal is detected, so that the gradient echo sequence is in the steady-state, and the acquisition of the gradient echo magnetic resonance signal is carried out while the gradient echo sequence is in the steady-state. This not only enables the acquisition of the gradient echo magnetic resonance signal when the gradient echo sequence is in a steady state, which improves the quality of the imaging, but also eliminates the need to perform a sufficient number of dummy scans from the beginning each time the trigger signal is detected to achieve the steady-state of the gradient echo sequence, which can greatly save the time before the steady-state acquisition, thus greatly saving the time of the MRI process.

In some embodiments, after acquiring the gradient echo magnetic resonance signal of the object (e.g., after the steady-state acquisition phase has ended), the computer device performs the next dummy scan of the object until the next trigger signal is detected.

The next dummy scan is a dummy scan performed after the completion of an acquisition of the gradient echo magnetic resonance signal.

In some embodiments, after completing the acquisition of the gradient echo magnetic resonance signal and before detecting the next trigger signal, the computer device controls the MRI device to perform the next dummy scan on the object immediately. In this embodiment, the purpose of the next dummy scan is to maintain the steady-state of the gradient echo sequence.

In some embodiments, after the next trigger signal is detected, the computer device then sequentially performs the steps described above after the trigger signal is detected, including: stopping the next dummy scan and sending the control signal to the MRI device to cause the MRI device to acquire a next gradient echo magnetic resonance signal of the object until a next data acquisition process is completed. Or, the computer device performs the steps including performing the dummy scan on the object during the trigger delay phase, and sending the control signal to the MRI device after the trigger delay phase has ended to cause the MRI device to acquire the gradient echo magnetic resonance signal of the object until the data acquisition process is completed.

In some embodiments, a determination of whether the data acquisition process is completed includes, at least one of: all gradient echo magnetic resonance signals of the object being acquired, a total duration of the gradient echo sequence being greater than a duration threshold, an amount of data acquired for the gradient echo magnetic resonance signals being greater than a data volume threshold, or the like. In some embodiments, the duration threshold and the data volume threshold is a system default value, an empirical value, a human pre-set value, or the like, or any combination thereof, which may be set according to the actual needs, and there is no limitation thereon.

As shown in FIG. 11, 114 denotes a next dummy scan phase, 115 denotes a current trigger signal, and 116 denotes a next trigger signal. At the end of the steady-state acquisition phase 113 and until the next trigger signal 116 is detected, the computer device sends the control signal to the MRI device to control the MRI device to continually perform the next dummy scan 114. After the computer device detects the next trigger signal 116, the computer device sends the control signal to the MRI device to proceed directly to the next steady-state acquisition phase, or to first proceed to the next trigger delay phase for the object to perform the delay dummy scan, and after the next trigger delay phase ends, the next steady-state acquisition phase is entered, and the gradient echo magnetic resonance signal of the object is acquired.

In some embodiments of the present disclosure, after acquiring the gradient echo magnetic resonance signal of the object, the next dummy scan on the object is continuously performed to maintain the steady-state of the gradient echo sequence, so that the gradient echo magnetic resonance signal may be acquired subsequently in the case of the steady-state of the gradient echo sequence, thereby improving the quality of the acquired gradient echo magnetic resonance signal, which in turn may improve the quality of the magnetic resonance image. At the same time, since the steady-state of the gradient echo sequence is maintained, it is not necessary to perform enough number of dummy scans (e.g., repeating the execution of 510) from the beginning in order to re-enter the steady-state of the gradient echo sequence, and thus it is possible to shorten the time of the MRI process and to improve the efficiency of the acquisition of the gradient echo magnetic resonance signal.

In some embodiments, during the process of the next dummy scan on the object, the computer device, in response to determining that a scanning feature of a current phase satisfies a first preset condition, triggers a stop instruction.

The current phase refers to the next dummy scan phase. The scanning feature of the current phase refers to the scanning feature when the next dummy scan is currently performed.

The scanning feature of the current phase includes temperature feature of the MRI device in the current phase, motion feature of the object in the current phase (e.g., motion frequency of the object, etc.), or the like. The temperature feature includes a temperature in a scanning channel of the MRI device, a coil temperature, or the like. See step 530 and its associated description for more description of the motion feature.

In some embodiments, the computer device obtains the temperature feature of the MRI device via a temperature sensor.

The first preset condition is a condition for determining whether to stop the next dummy scan. In some embodiments, the first preset condition includes the temperature of the MRI device (e.g., the temperature of the scanning channel and/or the temperature of the coil) being greater than a temperature threshold, the motion frequency of the object being greater than a frequency threshold, or the like. The scanning channel and the coil may correspond to different temperature thresholds.

In some embodiments, the temperature threshold and the frequency threshold is a system default value, an empirical value, a human pre-set value, or the like, or any combination thereof, which can be set according to the actual needs, and the present disclosure does not limit this.

In some embodiments, the computer device determines the temperature threshold based on an average value of historical temperatures of the MRI device (e.g., historical temperatures of the scanning channel and/or historical temperatures of the coil) when patients actively called off historical scans in the historical data. In some embodiments, the computer device determines the frequency threshold based on historical motion frequency of patients when the patients actively called off the historical scans in historical data.

The stop instruction is an instruction for controlling the MRI device to stop the next dummy scan. In some embodiments, when the scanning feature of the current phase satisfies the first preset condition, the computer device generates and sends the stop instruction to the MRI device to cause the MRI device to stop the dummy scan. For example, when the temperature of the scanning channel and/or the coil of the MRI device is greater than a corresponding temperature threshold, the computer device generates and sends the stop instruction to the MRI device to cause the MRI device to stop the dummy scan. For another example, when the motion frequency of the object is greater than the frequency threshold, the computer device generates and sends the stop instruction to the MRI device to cause the MRI device to stop the dummy scan.

In some embodiments, the computer device determines, based on the scanning feature of the current phase, a scanning feature of a next phase. In response to determining that the scanning feature of the next phase satisfies the first preset condition, the computer device triggers the stop instruction.

In some embodiments, when the current phase is the next dummy scan phase, the next phase may include one of the next trigger delay phase, the next steady-state acquisition phase, the next dummy scan phase following the next steady-state acquisition phase, or the like, or any combination thereof.

In some embodiments, the computer device performs a polynomial fit on the scanning features over a period of time in the past before the current time point to obtain a curve of the scanning feature over time. The scanning feature of the next phase is determined based on the curve of the scanning feature.

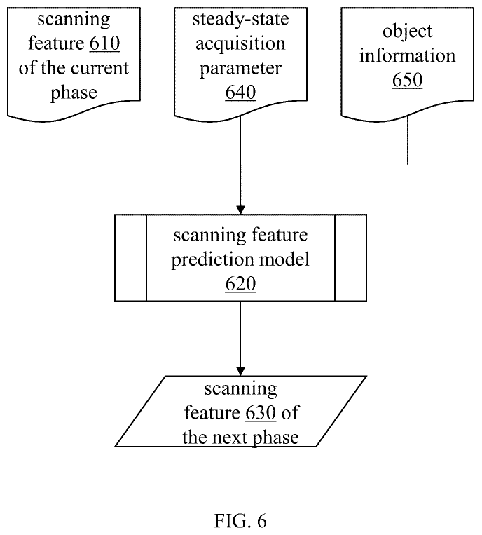

In some embodiments, the computer device determines the scanning feature of the next phase based on a scanning feature prediction model. For more on the scanning feature prediction model, see the FIG. 6 related description.

In some embodiments, the computer device, in response to the scanning feature of the next phase satisfying the first preset condition, generates and sends the stop instruction to the MRI device to cause the MRI device to stop the dummy scan. For example, when the temperature of the scanning channel and/or the temperature of the coil of the MRI device in the scanning feature of the next phase is greater than the corresponding temperature threshold, the computer device generates and sends the stop instruction to the MRI device to cause the MRI device to stop the dummy scan. For another example, when the motion frequency of the object in the scanning feature of the next phase is greater than the frequency threshold, the computer device generates and sends the stop instruction to the MRI device to cause the MRI device to stop the dummy scan.

Some embodiments of the present disclosure provide real-time monitoring of the dummy scan in terms of the scanning feature, which can stop the scan in a timely manner in the event of adverse effects on the patient to safeguard the personal safety of the patient. Based on the scanning feature of the current phase to predict the scanning feature of the next phase, when the scanning feature of the next phase does not satisfy the requirement, the next phase may be determined in advance, and the scan can be stopped in a timely manner, to further safeguard the personal safety of the patient.

In some embodiments, after the next dummy scan is stopped, in response to determining that a scanning feature of a last phase satisfies the first preset condition and a stopping time duration satisfies a second preset condition, the computer device triggers a start scanning instruction.

In some embodiments, the last phase is a phase between the current time point and a stopping time point of the dummy scan of the last phase.

The stopping time duration is a length of time from the stopping time point of the dummy scan of the last phase to the current time point.

The second preset condition is a condition that the stopping time duration needs to be satisfied when generating the start scanning instruction. In some embodiments, the second preset condition may include the stopping time duration being greater than a time threshold, etc.

In some embodiments, the time threshold is a system default, an empirical value, a human pre-set value, or the like. In some embodiments, the computer device determines an average value of historical time intervals each of which between a time point when the dummy scan was stopped and a time point when the dummy scan was restarted in the historical data as the time threshold.

The start scanning instruction is an instruction configured to control the MRI device to restart the dummy scan.

In some embodiments, in response to determining that the scanning feature of a last phase does not satisfy the first preset condition and the stopping time duration satisfies the second preset condition, the computer device generates and sends the start scanning instruction to the MRI device to cause the MRI device to restart the dummy scan. After restarting the dummy scan, the computer device enters the trigger delay phase or the steady-state acquisition phase in response to detecting the trigger signal. See the previous related description for more description.

Some embodiments of the present disclosure monitor in real time during the process of stopping the dummy scan, and restart the dummy scan at the appropriate time, which can shorten the stopping time duration and improve efficiency.

In some embodiments, the computer device performs the next dummy scan on the object after the first preset length of time until receiving the next trigger signal.

In some embodiments, after completing the acquisition of the gradient echo magnetic resonance signal and before the next trigger signal is detected, the computing device controls the MRI device not to perform the next dummy scan on the object immediately, but rather to perform the next dummy scan on the object after the first preset length of time. After detecting the next trigger signal, the computer device then sequentially performs the steps described above after detecting the trigger signal, including: stopping the next dummy scan, and sending the control signal to the MRI device to cause the MRI device to acquire the gradient echo magnetic resonance signal of the object until the data acquisition process is completed. Or, the computer device performs the steps including: performing the dummy scan on the object during the trigger delay phase, and sending the control signal to the MRI device at the end of the trigger delay phase to cause the MRI device to acquire the gradient echo magnetic resonance signal of the object until the data acquisition process is completed.

See the previous description for more information on the judgment condition for completion of the data acquisition task.

The first preset length of time is a time interval from a completion time point of the acquisition of the gradient echo magnetic resonance signal to a start time point of the next dummy scan.

In some embodiments, the first preset length of time is preset by the user based on historical experience.

In some embodiments, the computer device determines the first preset length of time based on a historical trigger interval and a triggering time point of a previous trigger signal. The historical trigger interval is an interval length between two adjacent trigger signals. In some embodiments, the computer device determines an interval length between current trigger signal and the previous trigger signal as the historical trigger interval. In some embodiments, the computer device determines an average interval length of a plurality of interval lengths as the historical trigger interval. Each of the plurality of interval lengths is an interval length between any two adjacent trigger signals.

After the acquisition of the gradient echo magnetic resonance signal is completed, in order to reduce the energy loss, instead of directly performing the next dummy scan, the object may be subjected to the next dummy scan after a certain period of time. It is only necessary to make sure that the next dummy scan and delay dummy scan within the trigger delay phase may ensure that the gradient echo sequence is in the steady-state in the next steady-state acquisition phase.

In some embodiments, the computer device determines the first preset length of time based on a triggering time point of the next trigger signal, a duration of a trigger delay phase corresponding to the trigger signal, and a steady-state recovery time.

In some embodiments, the computer device determines, based on a triggering time point corresponding to a historical trigger signal of the object, a time interval between two adjacent historical trigger signals, and determines, based on the mean value of the time intervals, a triggering time point of a current trigger signal, the triggering time point of the next trigger signal.

At the end of the steady-state acquisition phase, the steady-state of the gradient echo sequence will gradually dissipate if there is no next dummy scan performed. The steady-state recovery time is a time period it takes for the gradient echo sequence to change from unsteady-state to the steady-state.

In some embodiments, the computer device determines a current steady-state recovery time based on a plurality of historical steady-state recovery times after historical steady-state acquisition phases. For example, the computer device determines an average of the plurality of historical steady-state recovery times as the current steady-state recovery time.

See step 530 and its associated description for more description on the duration of the trigger delay phase.

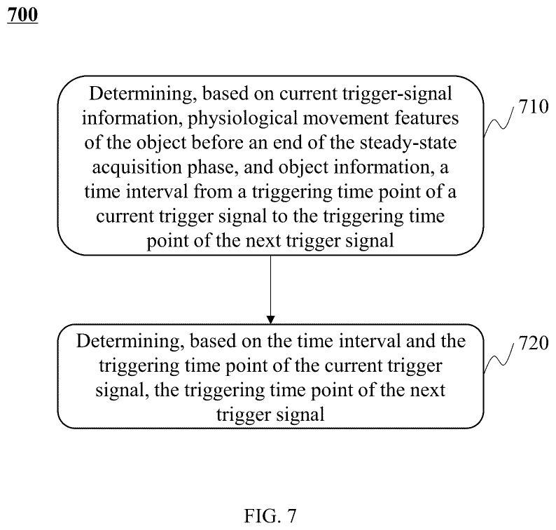

In some embodiments, the computer device determines the first preset length of time based on the current time point, the triggering time point of the next trigger signal, the duration of the trigger delay phase corresponding to the trigger signal, and the steady-state recovery time of the gradient echo sequence. Specifically, the first preset length of time=the triggering time point of the next trigger signal—the current time point—the steady-state recovery time+the duration of the trigger delay phase. For more on determining the triggering time point of the next trigger signal, see FIG. 7 and its related description.

In some embodiments of the disclosure, after the acquisition of the gradient echo magnetic resonance signal is completed, the next dummy scan is started after a period of time. The energy loss is reduced, environmentally-friendly while does not affect the scanning efficiency.

FIG. 6 is an exemplary schematic diagram of a scanning feature prediction model according to some embodiments of the present disclosure.

As shown in FIG. 6, in some embodiments, the processor processes a scanning feature 610 of a current phase based on the scanning feature prediction model 620 to determine a scanning feature 630 of a next phase. See FIG. 5 for more on the scanning feature of the current phase, and the scanning feature of the next phase in the related description.

The scanning feature prediction model is a model or algorithm configured to predict the scanning feature of the next phase. In some embodiments, the scanning feature prediction model is a machine learning model. For example, the scanning feature prediction model includes any one or a combination of a Deep-Learning Neural Network (DNN) model, a Convolutional Neural Networks (CNN) model, a Neural Networks (Neural Networks (NN) model or any one or combination of other customized model structures.

In some embodiments, as shown in FIG. 6, the input to the scanning feature prediction model 620 includes the scanning feature 610 of the current phase and the output is the scanning features 630 of the next phase.

In some embodiments, the scanning feature prediction model is obtained by training based on a plurality of first training samples with first labels. The computer device performs the following training process to obtain the scanning feature prediction model. The training process includes: obtaining the plurality of first training samples with first labels to form a first training sample set, and performing a plurality of iterations based on the first training sample set. At least one iterations comprises: selecting one or more first training samples from the training sample set, inputting the one or more first training samples into an initial scanning feature prediction model, obtaining model prediction outputs corresponding to the one or more first training samples; substituting the model prediction outputs corresponding to the one or more first training samples, and the first labels corresponding to the one or more first training samples, into a formula for a predefined loss function, calculating a value of the loss function; iteratively updating, based on the value of the loss function, model parameters in the initial scanning feature prediction model until an iteration end condition is satisfied, ending the iteration, and a trained scanning feature prediction model is obtained. The iterative updating the parameters of the initial scanning feature prediction model may be carried out by a variety of methods (e.g., a gradient descent method). The iteration end condition may include that the loss function converges or a number of iterations is greater than an iteration count threshold, or the like.

In some embodiments, the first training sample includes a scanning feature of a sample phase, and the first label is a scanning feature of a next phase of the sample phase. The processor may identify, in the historical data, a historical scanning feature of a historical phase as the first training sample, and identify a historical scanning feature of a next phase of that historical phase as the first label corresponding to the first training sample.

In some embodiments, when the next phase is a next dummy scan phase following a next steady-state acquisition phase, the input to the scanning feature prediction model 620 also includes a steady-state acquisition parameter 640, as shown in FIG. 6.

The steady-state acquisition parameter is a parameter related to the steady-state acquisition phase. For example, the steady-state acquisition parameter includes a duration of the steady-state acquisition phase, a number of data acquisitions, or the like.

In some implementations, the steady-state acquisition parameter is obtained via user input. For example, the user uploads the steady-state acquisition parameter via an input device.

In some embodiments, the steady-state acquisition parameters is accessed from a memory. The memory may be a memory that comes with an MRI device, or may be an external memory that is not part of the MRI device, such as, a hard disk, a CD-ROM, or the like. In some embodiments, the steady-state acquisition parameter is accessed via an interface, the interface including, but not limited to, a program interface, a data interface, a transmission interface, or the like. In some embodiments, the MRI device operates by automatically extracting the steady-state acquisition parameter from the interface. In some embodiments, the steady-state acquisition parameter is obtained in any manner known to those skilled in the art, and this disclosure does not limit this.

In some embodiments, when the input to the scanning feature prediction model includes the steady-state acquisition parameter, the first training sample also includes a sample steady-state acquisition parameter. The sample steady-state acquisition parameter may be acquired based on historical data from the same historical scanning process to which the scanning feature of the sample phase belongs.

In some embodiments of the present disclosure, when the next phase is the next dummy scan phase after the steady-state acquisition phase, when predicting the scanning feature of the next phase, the steady-state acquisition parameter is taken into account in consideration of the steady-state acquisition phase between the current phase and the next phase, as the input to the scanning feature prediction model. Thus, the obtained scanning feature of the next phase is accurate.

In some embodiments, the input to the scanning feature prediction model 620 also includes object information 650, as shown in FIG. 6.

The object information is data related to the object. For example, the object information includes an age, a gender, a disease type, or the like of the object. In some embodiments, the processor determines the object information based on medical record file of the object. The medical record file is obtained in the same manner as the steady-state acquisition parameter, and is not described herein.

In some embodiments, when the input to the scanning feature prediction model includes the object information, the first training sample also includes sample object information. The sample object information corresponds to a sample object that is the historical object that is targeted by the same historical scanning process to which the sample steady-state acquisition parameter and the scanning feature of the sample phase belongs to, and the sample object information may be obtained based on the historical data.

In some embodiments of the present disclosure, when predicting the scanning feature of the next phase, actual situation of the patient is taken into account, and the obtained scanning feature of the next phase can be tailored to the actual situation of the patient.

In some embodiments, the processor divides the plurality of first training samples with the first labels in the first training sample set into a training subset, a testing subset, and a validation subset, respectively. Each subset includes a plurality of first training samples with the first labels. The computer device may train the scanning feature prediction model based on the training subset, the testing subset, and the validation subset by a cross-validation method. The cross-validation method includes, but is not limited to, a hierarchical cross-validation algorithm, a repeated cross-validation algorithm, or the like.

In some embodiments, the training subset is a dataset configured to adjust learning parameters of the scanning feature prediction model during model training. The learning parameters include parameters such as a weight, a bias, or the like. The validation subset is a dataset configured to adjust hyperparameters of the scanning feature prediction model during model training. The hyperparameters include a number of network layers, a number of network nodes, a number of iterations, a learning rate, or the like. The testing subset is a dataset configured to evaluate the performance of the final scanning feature prediction model.

In some embodiments, a number of samples in the training subset, a number of samples in the validation subset, and a number of samples in the testing subset satisfy a preset ratio. The number of samples is a number of first training samples.

In some embodiments, the preset ratio is set by a human being. For example, the preset ratio is 8:1:1.

In some embodiments, the training subset, the testing subset, and the validation subset have no data crossover.

No data crossover refers that the same piece of data (e.g., a first training sample with the first label) can only exist in one of the training subset, testing subset, and validation subset.

In some embodiments, a sample statistic difference of the training subset is greater than a preset difference threshold.

The sample statistic difference is an indicator that reflects the sample diversity of the training subset, the greater the sample diversity, the greater the sample statistic difference. The sample diversity of the training subset is the diversity of the plurality of first training samples contained in the training subset.

In some embodiments, the processor vectorizes each of the first training samples in the training subset. For example, the computer device quantifies the scanning feature of the sample phase in the first training sample and constructs a feature vector. For example, the computer device quantifies the sample object information, the scanning feature of the sample phase in the first training sample, and constructs the feature vector. For example, the computer device quantifies the sample steady-state acquisition parameter, the scanning feature of the sample phase in the first training sample, and constructs the feature vector. For another example, the computer device quantifies the sample object information, the sample steady-state acquisition parameter, and the scanning feature of the sample phase in the first training sample, and constructs the feature vector.

In some embodiments, the computer device determines a vector distance between feature vectors corresponding to each of the two first training samples in the training subset, calculates a statistical value (e.g., a variance) for all of the vector distances, and identifies the statistical value as the sample statistic difference. The vector distance may be a Euclidean distance, a cosine distance, or the like.

The preset difference threshold is a threshold for determining whether the sample statistic difference satisfies a requirement. In some embodiments, the preset difference threshold is pre-set based on historical data or prior knowledge. In some embodiments, the processor determines the preset difference threshold based on historical data. For example, the preset difference threshold is positively correlated with a magnitude of fluctuations in image quality of a plurality of historical magnetic resonance images in the historical data, and the magnitude of fluctuations may be expressed as a coefficient of variation of image quality of the plurality of historical magnetic resonance images. For more on image quality see FIG. 5 and related notes.

In some embodiments, the computer device trains the scanning feature prediction model based on the training subset; after training based on the training subset, the computer device verifies based on the validation subset; and after verifying based on the validation subset, the computer device tests the scanning feature prediction model based on the testing subset.

In some embodiments, the computer device performs at least one iteration of training based on the training subset to obtain a scanning feature prediction model with determined learning parameters.

In some embodiments, the computer device validates the scanning feature prediction model with the determined learning parameters based on the validation subset, and adjusts hyperparameters of the scanning feature prediction model with the determined learning parameters based on a validation result.

In some embodiments, the computer device tests the scanning feature prediction model with determined learning parameters and hyperparameters based on the testing subset to assess a generalization capability of the scanning feature prediction model.

The embodiments of the present disclosure do not have any special limitation on the way of model training using the training subset, the testing subset, and the validation subset, and it is sufficient to adopt the operations known to those skilled in the art.

Some embodiments of the present disclosure, by limiting the sample statistic difference in the testing set, are able to make the model robust, prevent the model from overfitting, and improve the accuracy of the model. The greater the volatility of the historically acquired effects, the more unstable the quality of the images obtained from the reconstruction of historically acquired gradient echo magnetic resonance signals, and the more potential influences of the various aspects involved. Therefore, the preset variance thresholds may be tuned up to allow the effect determination model to learn from a plurality of widely distributed data samples to more accurately learn the prediction of the target. The use of cross-validation to train the scanning feature prediction model can improve the stability and accuracy of the model.

Some embodiments of the present disclosure, by predicting the scanning feature of the next phase through the scanning feature prediction model, utilizes the self-learning capability of a machine learning model to find a law from a large amount of historical data, and obtain a correlation relationship between the scanning feature of the current phase and the scanning feature of the next phase, improving the accuracy and efficiency of determining the scanning feature of the next phase, so as to effectively determine whether to stop the dummy scan based on the scanning feature of the next phase. Thus, the safety of the patient can be further ensured.

In some embodiments, the computer device determines a time interval from a triggering time point of a current trigger signal to the triggering time point of the next trigger signal based on current trigger-signal information, physiological movement features of the object before an end of the steady-state acquisition phase, and object information. The computer device determines, based on the time interval and the triggering time point of the current trigger signal, the triggering time point of the next trigger signal.

FIG. 7 is an exemplary flowchart for determining a triggering time point of a next trigger signal according to some embodiments of the present disclosure.

In some embodiments, process 700 may be executed by a computer device. For example, the process 700 is stored in a memory in the form of a program or instruction, and the process 700 is implemented when the computer device executes the program or instruction. Schematic diagram of the operation of the process 700 presented below is illustrative. In some embodiments, one or more additional operations not described and/or one or more operations not discussed may be utilized to complete the process. Also, the order of the operations of the process 700 illustrated in FIG. 7 and described below is not limiting.

Step 710, a time interval from a triggering time point of a current trigger signal to the triggering time point of the next trigger signal is determined based on current trigger-signal information, physiological movement features of the object before an end of the steady-state acquisition phase, and object information.

See FIG. 5 related descriptions of the physiological movement feature and FIG. 6 related descriptions of the object information.

The current trigger-signal information refers information related to the current trigger signal. For example, the current trigger-signal information includes the triggering time point of the current trigger signal, a physiological signal, a motion sensing signal, or the like corresponding to the triggering time point of the current trigger signal. For more on the physiological signal and the motion sensing signal, see FIG. 5 and its related description.

The physiological movement feature of the object before the end of the steady-state acquisition phase refers a physiological movement feature of the object between the triggering time point of the current trigger signal and a completion time point of the acquisition of the gradient echo magnetic resonance signal.

In some embodiments, the computer device constructs a clustering vector, based on historical trigger-signal information, a historical physiological movement feature of a historical object before the end of the steady-state acquisition phase, historical object information, and a time interval between a triggering time point of a previous historical trigger signal and a triggering time point of a next historical trigger signal in the historical data. The computer device clusters the clustering vectors to obtain a plurality of clustering centers. The computer device constructs standard vectors based on the historical trigger-signal information corresponding to the plurality of clustering centers, the historical physiological movement features of the historical object before the end of the steady-state acquisition phase, and the historical object information. The computer device determines time intervals from the triggering time point of the previous historical trigger signal to the triggering time point of the next historical trigger signal corresponding to the clustering centers as labels of the standard vectors. The computer device constructs a standard vector library based on the standard vectors and labels corresponding to the plurality of clustering centers. The clustering method includes, but is not limited to, a K-Means clustering algorithm, a DBSCAN clustering algorithm, or the like, and a number of clustering centers may be artificially preset.

In some embodiments, the computer device constructs a to-be-matched vector based on the current trigger-signal information, the physiological movement feature of the object before the end of the steady-state acquisition phase, and the object information. The computer device matches the to-be-matched vector in the standard vector library, and determines a label corresponding to a standard vector that has a smallest vector distance from the to-be-matched vector as the time interval from the triggering time point of the current trigger signal to the triggering time point of the next trigger signal. The vector distance may include, but is not limited to, a Euclidean distance, a cosine distance, or the like.

In some embodiments, the computer device determines, based on the current trigger-signal information, the physiological movement feature of the object before the end of the steady-state acquisition phase, and the object information, by a time interval prediction model, the time interval from the triggering time point of the current trigger signal to the triggering time point of the next trigger signal.

The time interval prediction model is a model for predicting the time interval from the triggering time point of the current trigger signal to the triggering time point of the next trigger signal. In some embodiments, the time interval prediction model is a machine learning model, e.g., any one or a combination of, for example, a DNN model, a CNN model, an NN model, or other customized model structures.

In some embodiments, inputs to the time interval prediction model include the current trigger-signal information, the physiological movement features of the object before the end of the steady-state acquisition phase, and the object information, and output of the time interval prediction model is the time interval from the triggering time point of the current trigger signal to the triggering time point of the next trigger signal.

In some embodiments, the time interval prediction model is obtained by training a plurality of second training samples with second labels. The computer device may perform the following training process to obtain the time interval prediction model. The training process includes: obtaining the plurality of second training samples with second labels to form a second training sample set, and performing a plurality of rounds of iterations based on the second training sample set. The at least one iteration includes: selecting one or more second training samples from the second training sample set, inputting the one or more second training samples into an initial time interval prediction model, obtaining one or more model prediction outputs corresponding to the one or more second training samples; substituting the model prediction outputs corresponding to the one or more second training samples, and the second labels corresponding to the one or more second training samples, into a formula for a predefined loss function, calculating a value of the loss function; iteratively updating, based on the value of the loss function, model parameters in the initial time interval prediction model until an iteration end condition is satisfied, ending the iteration, and a trained time interval prediction model is obtain. The iterative updating of the parameters of the initial time interval prediction model may be carried out by a variety of methods (e.g., a gradient descent method). The iteration end condition may include that the loss function converges or the number of iterations is greater than an iteration count threshold, or the like.

In some embodiments, a second training sample includes sample trigger signal information, sample physiological movement features of a sample object before the end of the steady-state acquisition phase, and sample object information. A second label is a time interval between a triggering time point of the sample trigger signal corresponding to the second training sample and the triggering time point of the next trigger signal. The second training samples and the second labels may be determined based on historical data.

In some embodiments of the present disclosure, a time interval between two adjacent trigger signals is predicted by the time interval prediction model, and the prediction result is accurate.

Step 720, the triggering time point of the next trigger signal is determined based on the time interval and the triggering time point of the current trigger signal.

In some embodiments, the computer device determines the triggering time point of the next trigger signal based on the time interval from the triggering time point of the current trigger signal to the triggering time point of the next trigger signal and the triggering time point of the current trigger signal. For example, the triggering time point of the next trigger signal=the triggering time point of the current trigger signal+the time interval from the triggering time point of the current trigger signal to the triggering time point of the next trigger signal.

Some embodiments of the present disclosure determine the triggering time point of the next trigger signal based on the current trigger-signal information, the physiological movement features of the object before the end of the steady-state acquisition phase, and the object information. The triggering time point of the next trigger signal obtained by a prediction process described above is close to the actual situation.

In some embodiments, the computer device sends the control signal to an MRI device to cause the MRI device to perform a dummy scan on the object includes that: the computer device sends the control signal to the MRI device to cause the MRI device to apply a first radio frequency pulse to the object and simultaneously apply a first selective slice gradient field to the object without acquiring signals.

In some embodiments, the computer device sends the control signal to the MRI device to cause the MRI device to perform the dummy scan on the object also includes that: the computer device sends the control signal to the MRI device to cause the MRI device to apply a first dephasing gradient field to the object after applying the first selective slice gradient field to the object.

FIG. 8 is an exemplary flowchart for performing a dummy scan on an object according to some embodiments of the present disclosure.

In some embodiments, a process 800 is executed by a computer device. For example, the process 800 is stored in a memory in the form of a program or instruction, and when the computer device executes the program or instruction, the process 800 is implemented. The schematic representation of the operation of the process 800 presented below is illustrative. In some embodiments, one or more additional operations not described and/or one or more operations not discussed may be utilized to complete the process. Additionally, the order of the operations of the process 800 illustrated in FIG. 8 and described below is not limiting. It is to be noted that there is no necessary sequence or other relationship between step 810 and step 820. For example, the computer device may perform only the operation of the step 810 during a dummy scan phase, or it may perform the operation of step the 810 before performing the operation of the step 820 during the dummy scan phase.

Step 810, a first radio frequency pulse is applied to the object and simultaneously a first selective slice gradient field is applied to the object without acquiring signals.

The first radio frequency pulse is a radio frequency pulse applied during the dummy scan phase. In some embodiments, the first radio frequency pulse is excited at an angle between 0° and 90°. For example, the first radio frequency pulse is excited at an angle between 10° and 90°.

In some embodiments, an MRI device includes three pairs of gradient coils that can generate gradient magnetic fields in three directions, respectively. The three pairs of gradient coils correspond to X-axis, Y-axis, and Z-axis of a spatial right-angled coordinate system. The Z-axis direction is the same as a direction of an applied magnetic field, and the X-axis and the Y-axis are perpendicular to the Z-axis direction. By energizing the gradient coils, gradient fields can be generated in the corresponding coordinate axes (in the X-axis direction, Y-axis direction, and Z-axis direction), respectively.

In the sequence diagram, a waveform of the gradient field may be approximated as a trapezoid, including a rising edge, a plateau period, and a falling edge. When energizing the gradient coil, the magnetic field strength gradually rises, corresponding to the rising edge; when the magnetic field strength reaches the maximum, the magnetic field strength remains stable for a period of time, corresponding to the plateau period; and when stopping the energization of the gradient coil, the magnetic field strength gradually decreases to 0, corresponding to the falling edge. By changing a direction and a magnitude of a current input to the gradient coil, the direction of the magnetic field and the maximum magnetic field strength of the gradient field may be changed.

The first selective slice gradient field is a selective slice gradient field applied during the dummy scan phase. The selective slice gradient field is a gradient field configured to select a slice in space. A selective slice gradient field is applied to selectively excite a certain slice under the action of a radio frequency pulse.

In some embodiments, applying the first selective slice gradient field to the object includes applying the gradient field to a target axis. The target axis is any one or combination of the three coordinate axes of the X-axis, the Y-axis, and the Z-axis.

In some embodiments, the computer device sends a control signal to the MRI device to cause the MRI device to apply the first radio frequency pulse to the object and simultaneously apply the first selective slice gradient field to the object. That is, the start time of the first radio frequency pulse is the same as the start time of the plateau period of the first selective slice gradient field, and the end time of the first radio frequency pulse is the same as the end time of the plateau period of the first selective slice gradient field.

The present embodiment does not limit the form of the first selective slice gradient field.

In some embodiments, the computer device sends the control signal to the MRI device to cause the MRI device to not acquire a gradient echo magnetic resonance signal of the object after applying the first radio frequency pulse and the first selective slice gradient field.

In some embodiments of the present disclosure, applying the first selective slice gradient field at the same time as applying the first radio frequency pulse may ensure that the first radio frequency pulse resonates with a specific slice (the slice corresponding to the first selective slice gradient field) to produce the gradient echo magnetic resonance signal. The dummy scan on the object is achieved by controlling the MRI device to apply the first radio frequency pulse and the first selective slice gradient field to the object. This way of performing the dummy scan on the object is simple and easy to implement.

Step 820, a first dephasing gradient field is applied to the object after applying the first selective slice gradient field to the object.

The first dephasing gradient field is a dephasing gradient field configured to compensate for the first selective slice gradient field. A dephasing gradient may be formed by applying the gradient magnetic field to at least one of the X-axis, the Y-axis, and the Z-axis of the gradient coils in a gradient direction.

In some embodiments, the computer device sends the control signal to the MRI device to cause the MRI device to apply the first dephasing gradient field to the object after applying the first selective slice gradient field to the object. The target axis of the first dephasing gradient field may be any one or a combination of the three coordinate axes of the X-axis, the Y-axis, and the Z-axis.

The embodiments of the present disclosure do not limit the form of the first dephasing gradient field.

In some embodiments, the computer device sends the control signal to the MRI device to cause the MRI device to refocus the signals by applying, after applying the first selective slice gradient field, a negative gradient field in the direction of the slice corresponding to the first selective slice gradient field. The negative gradient field is a first refocusing gradient field. The first refocusing gradient field is a refocusing gradient field applied after the first selective slice gradient field is applied. A direction of the magnetic field of the first refocusing gradient field is opposite to a direction of the magnetic field of the first selective slice gradient field.

In some embodiments, the computer device sends the control signal to the MRI device to cause the MRI device to apply a negative first refocusing gradient field in the direction of the slice corresponding to the first selective slice gradient field, after applying the first selective slice gradient field. The computer device applies the first dephasing gradient field to the object while applying the first refocusing gradient field.

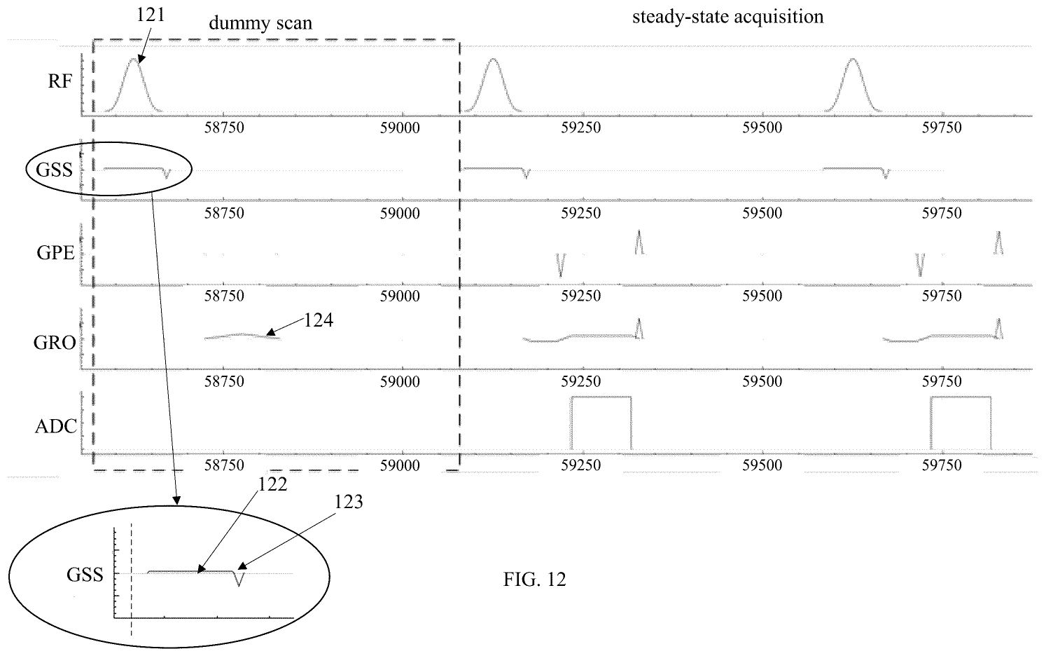

FIG. 12 is a sequence diagram from an MRI procedure shown in FIG. 12 is another sequence diagram during an MRI process according to some embodiments of the present disclosure. FIG. 12 contains a sequence diagram of a dummy scan on an object. As shown in the leftmost dashed box of FIG. 12, performing the dummy scan includes: applying a first radio frequency pulse 121 (corresponding to the first waveform on the RF-axis of FIG. 12) on the RF-axis, and simultaneously applying a first selective slice gradient field 122 (corresponding to the first trapezoidal portion on the GSS-axis of FIG. 12) on the GSS-axis to generate the gradient echo magnetic resonance signal. Then, after the first radio frequency pulse 121 has been applied, performing the dummy scan further includes: applying a first refocusing gradient field 123 (corresponding to the inverted triangle portion after the first selective slice gradient field 122 in FIG. 12) on the GSS-axis to refocus the signal; and finally applying a first dephasing gradient field 124 (corresponding to the first positive triangular portion on the GRO-axis of FIG. 12) on the GRO-axis. The first dephasing gradient field may be applied to at least one of the GSS-axis, the GPE-axis, and the GRO-axis.

More descriptions of symbols shown in FIG. 10, such as RF, GRO, GPE, GSS, ADC, and SYNC are described in FIG. 1 and its related descriptions.

The present embodiment does not limit the form of the first dephasing gradient field, the first refocusing gradient field, as long as it can realize its function.

In some embodiments of the present disclosure, applying the first dephasing gradient field to the object after applying the first selective slice gradient field to the object may remove current transverse magnetization vectors, which is capable of improving the quality of the acquired gradient echo magnetic resonance signal, and in turn the quality of the magnetic resonance image of the object is improved.

In some embodiments, the computer device sends the control signal to the MRI device to cause the MRI device to apply a second radio frequency pulse to the object and simultaneously apply a second selective slice gradient field to the object. The computer device applies a phase-encoded gradient field and a frequency-encoded gradient field to the object after a second preset length of time to acquire the gradient echo magnetic resonance signal of the object. For more on the gradient echo magnetic resonance signal, see FIG. 5 and its related instructions. FIG. 9 is an exemplary flowchart for acquiring a gradient echo magnetic resonance signal of an object according to some embodiments of the present disclosure.

In some embodiments, a process 900 is executed by a computer device. For example, the process 900 is stored in a memory in the form of a program or instruction, and the process 900 is implemented when the computer device executes the program or instruction. The schematic representation of the operation of the process 900 presented below is illustrative. In some embodiments, the process 900 may be accomplished utilizing one or more additional operations that are not described and/or one or more operations that are not discussed. Also, the order of the operations of the process 900 illustrated in FIG. 9 and described below is not limiting.

Step 910, a second radio frequency pulse is applied to the object and simultaneously a second selective slice gradient field is applied to the object.

The second radio frequency pulse is a radio frequency pulse applied during a steady-state acquisition phase. For more on the steady-state acquisition phase see FIG. 5 and its related instructions.

The second selective slice gradient field is a selective slice gradient field applied during the steady-state acquisition phase.

The second radio frequency pulse may be the same as or different from a first radio frequency pulse. The second selective slice gradient field may be the same as or different from a first selective slice gradient field. For more on the radio frequency pulse and the selective slice gradient field see FIG. 8 and its related instructions.

In some embodiments, in response to receiving a trigger signal, the computer device sends a control signal to an MRI device to cause the MRI device to apply the second radio frequency pulse to the object and simultaneously apply the second selective slice gradient field to the object. In some embodiments, the MRI device applies the second radio frequency pulse and the second selective slice gradient field by the same method as applying the first radio frequency pulse and the first selective slice gradient field, and a description of how the first radio frequency pulse and the first selective slice gradient field are applied is described in the FIG. 8 related description.

In some embodiments, the computer device sends the control signal to the MRI device to cause the MRI device to refocus the signal after applying the second selective slice gradient field by applying a negative second refocusing gradient field in the direction of the slice corresponding to the second selective slice gradient field. The second refocusing gradient field is a refocusing gradient field that is applied after the second selective slice gradient field is applied. The direction of the magnetic field of the second refocusing gradient field is opposite to the direction of the magnetic field of the second selective slice gradient field.

Step 920, a phase-encoded gradient field, and a frequency-encoded gradient field are applied to the object after a second preset length of time to acquire the gradient echo magnetic resonance signal of the object.

The second preset length of time is a duration between a point in time at which the second selective slice gradient field has finished functioning and a point in time at which the phase-encoded gradient field and the frequency-encoded gradient field begin to be applied. The second preset length of time may be preset by a user based on historical data or a priori knowledge.

The phase-encoded gradient field and the frequency-encoded gradient field are gradient fields configured for spatial localization. The phase-encoded gradient field and the frequency-encoded gradient field may be formed by applying gradient magnetic fields to at least one of the gradient directions in the X-axis, the Y-axis, and the Z-axis of the gradient coils.

In some embodiments, the computer device, while controlling the MRI device to apply the second selective slice gradient field to the object, sends the control signal to the MRI device to cause the MRI device to apply the phase-encoded gradient field and the frequency-encoded gradient field after the second preset length of time, and acquire the gradient echo magnetic resonance signal of the object with the phase-encoded gradient field and the frequency-encoded gradient field applied.

In some embodiments of the present disclosure, the gradient echo sequence applied to the object includes the second radio frequency pulse, the second selective slice gradient field, the phase-encoded gradient field, and the frequency-encoded gradient field. The gradient echo sequence is simple in structure and easy to implement.

In some embodiments, during acquisition of the gradient echo magnetic resonance signal of the object, the computer device also sends the control signal to the MRI device to cause the MRI device to apply a second dephasing gradient field to the object before applying the frequency-encoded gradient field to the object; and to cause the MRI device to apply a third dephasing gradient field to the object after applying the frequency-encoded gradient field to the object.

The second dephasing gradient field is a dephasing gradient field configured to compensate for the second selective slice gradient field.

The second dephasing gradient field may be applied to the object before applying the frequency-encoded gradient field to the object.

The second dephasing gradient field may be applied prior to applying the phase-encoded gradient field. The second dephasing gradient field may be applied after applying the phase-encoded gradient field. The second dephasing gradient field may be applied at the same time as the phase-encoded gradient field.

The second dephasing gradient field corresponds to the same target axis as the frequency-encoded gradient field corresponds to. The target axis corresponding to the second dephasing gradient field may be formed by applying a gradient magnetic field to at least one of the gradient directions in at least one of the X-axis, Y-axis, and Z-axis of the gradient coils.

The target axis corresponding to the third dephasing gradient field may be formed by applying a gradient magnetic field to at least one of the gradient directions in at least one of the X-axis, Y-axis, and Z-axis of the gradient coils. The second dephasing gradient field and the third dephasing gradient field may be in the same form or may be different.

As shown in FIG. 12, a steady-state acquisition comprises: a second radio frequency pulse is applied on the RF-axis (corresponding to the second and third waveforms on the RF-axis of FIG. 12); a second selective slice gradient field is applied on the GSS-axis (corresponding to the second and third trapezoidal portions on the GSS-axis of FIG. 12), and a second refocusing gradient field is applied after the second selective slice gradient field (corresponding to the inverted triangle portion after the second selective slice gradient field on the GSS-axis of FIG. 12); the phase-encoded gradient field is imposed on the GPE-axis (corresponding to the first and second negative triangular portions on the GPE-axis of FIG. 12); the second dephasing gradient field is imposed on the GRO-axis (corresponding to the first and second negative trapezoidal portions on the GRO-axis of FIG. 12), and the frequency-encoded gradient field is imposed on the GRO-axis (corresponding to the forward trapezoid after the second dephasing gradient field on the GRO-axis); the third dephasing gradient field (corresponding to the neighboring triangular portions on the GPE-axis and GRO-axis of FIG. 12) is applied on the GPE and GRO axes. The second dephasing gradient field is applied to the GRO-axis, and the third dephasing gradient field may be applied to at least one of the GPE-axis, the GRO-axis, and the GSS-axis. When applying the frequency-encoded gradient field, an analog-to-digital converter needs to be turned on to acquire the gradient echo magnetic resonance signal.

Some embodiments of the present disclosure, by applying the third dephasing gradient field, residual signals are eliminated and the accuracy of the acquired gradient echo magnetic resonance signal is improved, and thus the quality of the magnetic resonance images is improved.

The gradient echo sequence applied to the object as described in some embodiments of the present disclosure includes the second radio frequency pulse, the second selective slice gradient field, the phase-encoded gradient field, and the frequency-encoded gradient field. The gradient echo sequence is simple in structure and easy to implement.

In some embodiments, a single echo signal (as shown in FIG. 12) or a plurality of echo signals may be acquired simultaneously during applying the gradient echo sequence to the object. Acquisition of the plurality of echo signals may be used in multi-echo correlation applications such as fat quantification calculations.

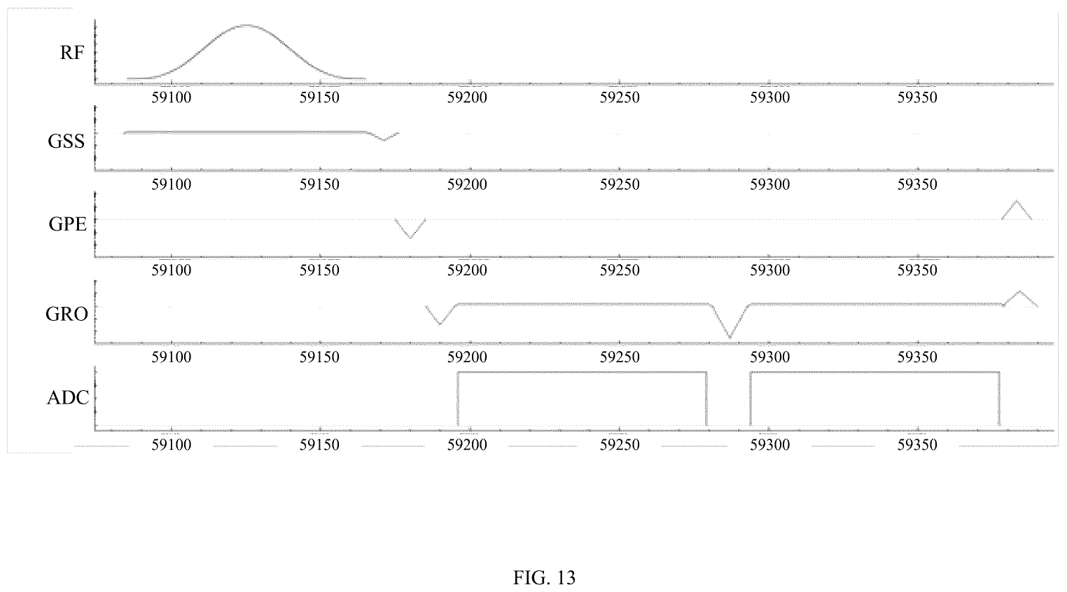

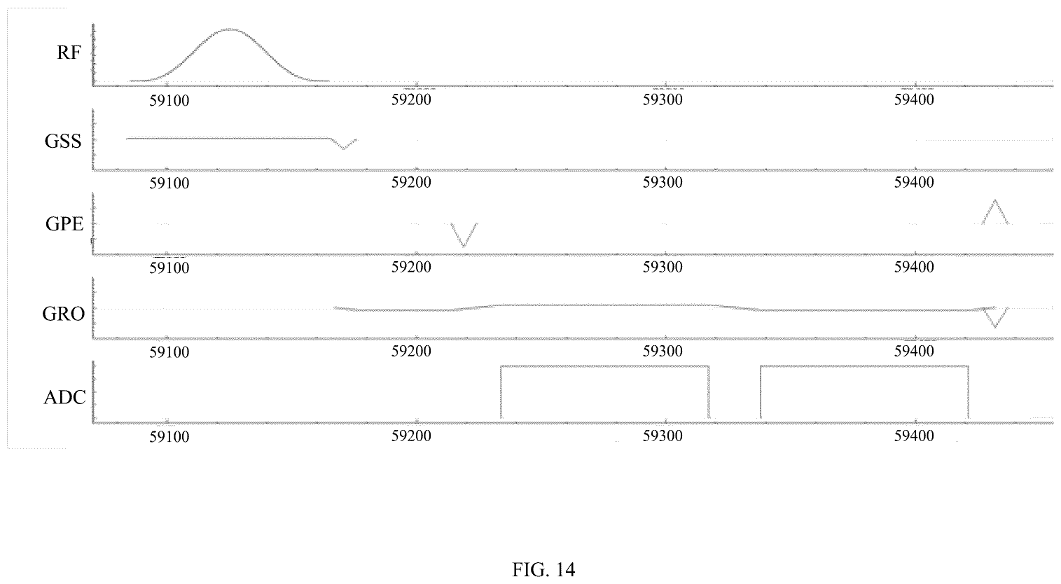

Acquisition of the plurality of echo signals consists of two acquisition modes, one is a same-polarity acquisition mode, as shown in FIG. 13, and the other is a reverse-polarity acquisition mode, as shown in FIG. 14. From FIG. 13, it can be seen that the same-polarity acquisition mode is to apply the frequency-encoded gradient fields of the same polarity on the GRO-axis (e.g., two positive trapezoidal on the GRO-axis, and there is a dephasing gradient field between two positive trapezoidal parts). From FIG. 14, it can be seen that the reverse-polarity acquisition mode is realized by applying the frequency-encoded gradient fields of opposite polarity on the GRO-axis (e.g., one positive gradient and one negative gradient on the GRO-axis).

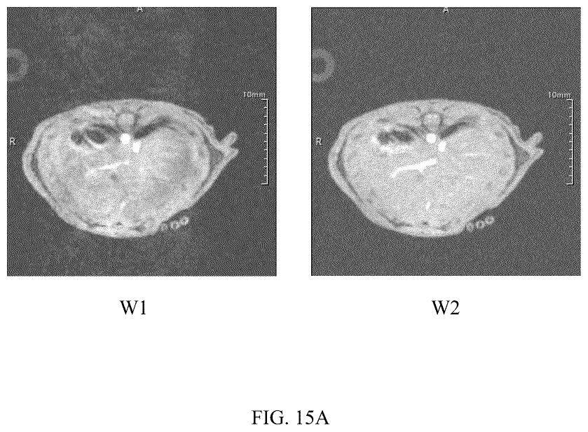

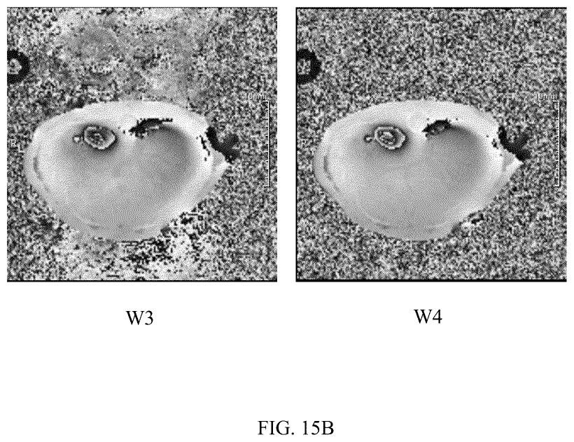

FIG. 15A is a magnitude image corresponding to a magnetic resonance image according to some embodiments of the present disclosure, and FIG. 15B is a phase image corresponding to a magnetic resonance image according to some embodiments of the present disclosure. W1 in FIG. 15A denotes a magnitude image corresponding to a magnetic resonance image obtained by acquiring and reconstructing a gradient echo magnetic resonance signal without a trigger signal. W2 in FIG. 15A denotes a magnitude image corresponding to the magnetic resonance image obtained by acquiring and reconstructing the gradient echo magnetic resonance signal within the trigger signal. W3 in FIG. 15B denotes a phase image corresponding to the magnetic resonance image obtained by acquiring and reconstructing a gradient echo magnetic resonance signal without a trigger signal. W4 in FIG. 15B denotes a phase image corresponding to the magnetic resonance image obtained by acquiring and reconstructing the gradient echo magnetic resonance signal within the trigger signal. It can be seen by comparison that the magnetic resonance image obtained by acquiring and reconstructing the gradient echo magnetic resonance signal without the trigger signal has obvious motion artifacts, while the magnetic resonance image obtained by using a method for MRI provided by the embodiments of the present disclosure has no obvious motion artifacts in the corresponding magnitude image and phase image.

Some embodiments of the present disclosure provide a method for MRI, the steps of which include:

Step S1, a dummy scan is performed on an object until a steady-state of a gradient echo sequence is reached, and the dummy scan is continued and the steady-state of the gradient echo sequence is maintained.

The dummy scan includes: applying a first radio frequency pulse, a first selective slice gradient field, a first refocusing gradient field, and a first dephasing gradient field to the object while without acquiring signals.

Step S2, in response to determining that no trigger signal is detected while the gradient echo sequence is in the steady-state, the dummy scan is continuously performed. In response to receiving the trigger signal, the dummy scan is performed on the object during a trigger delay phase corresponding to the trigger signal

Step S3, during the steady-state acquisition phase after the trigger delay phase ends, a second radio frequency pulse is applied to the object and simultaneously a second selective slice gradient field is applied to the object.

Step S4, a phase-encoded gradient field and a second dephasing gradient field are applied to the object after a second preset length of time

Step S5, a frequency-encoded gradient field is applied to the object after the second preset length of time and an analog-to-digital converter is turned on to acquire a gradient echo magnetic resonance signal, and a third dephasing gradient field is applied to the object.

Step S6, Steps S3-S5 are executed for a plurality of times, and a plurality of signal acquisitions are performed on the object until a number of data acquisitions in the steady-state acquisition phase is greater than a preset number.

Step S7, at the end of the steady-state acquisition phase, the dummy scan is continuously performed on the object to maintain the steady-state of the gradient echo sequence, and then return to perform steps S2-step S7 to acquire all gradient echo magnetic resonance signals of the object. A magnetic resonance image of the object is reconstructed based on the gradient echo magnetic resonance signals.

In some embodiments, a non-transitory computer readable medium storing instructions is provided. The instructions, when executed by at least one processor, causes the at least one processor to implement a method comprising: applying a gradient echo sequence to an object and performing a dummy scan on the object until a steady-state of the gradient echo sequence is reached; continuing the dummy scan and maintaining the steady-state of the gradient echo sequence; in response to detecting a trigger signal while the gradient echo sequence is in the steady-state, acquiring a gradient echo magnetic resonance signal of the object.

Some embodiments of the present disclosure further provide a computer program product comprising a computer program which, when executed by a processor, accomplishes the steps of: applying a gradient echo sequence to an object and performing a dummy scan on the object until a steady-state of the gradient echo sequence is reached; continuing the dummy scan and maintaining the steady-state of the gradient echo sequence; and in response to receiving a trigger signal while the gradient echo sequence is in the steady-state, acquiring a gradient echo magnetic resonance signal of the object.

A person of ordinary skill in the art may understand that realizing all or part of the processes in the methods of the above embodiments is possible by means of a computer program that instructs the relevant hardware to complete the same, and that the computer program may be stored in a non-volatile computer-readable storage medium, which computer program, when executed, may include processes such as those of the embodiments of the present disclosure. Any reference to a memory, database, or other medium used in the embodiments provided in the present disclosure may comprise at least one of a non-volatile memory and a volatile memory. The non-volatile memory may include read-only memory (ROM), magnetic tape, floppy disk, flash memory, optical memory, high-density embedded non-volatile memory, ReRAM, magnetoresistive random access memory (MRAM), ferroelectric random access memory (FRAM), phase change memory (PCM), graphene memory, or the like. The volatile memory may include random access memory (RAM) or external cache memory, or the like. As an illustration and not as a limitation, RAM may be in a plurality of forms, such as static random access memory (SRAM) or dynamic random access memory (DRAM), or the like. The databases involved in the embodiments provided in the present disclosure may include at least one of a relational database or a non-relational database. The non-relational databases may include a plurality of blockchain-based distributed databases or the like. Processors involved in the embodiments provided in the present disclosure may be general purpose processors, central processors, graphics processors, digital signal processors, programmable logic devices, quantum computing based data processing logic devices, or the like, without limitation.

The basic concepts have been described above, and it is apparent to those skilled in the art that the foregoing detailed disclosure serves only as an example and does not constitute a limitation of the present disclosure. While not expressly stated herein, a person skilled in the art may make various modifications, improvements, and amendments to this disclosure. Those types of modifications, improvements, and amendments are suggested in this disclosure, so those types of modifications, improvements, and amendments remain within the spirit and scope of the exemplary embodiments of this disclosure.

Also, the disclosure uses specific words to describe embodiments of the disclosure. Such as “an embodiment,” “an embodiment,” and/or “some embodiment” means a feature, structure, or characteristic associated with at least one embodiment of the present disclosure. Accordingly, it should be emphasized and noted that two or more references in this disclosure, at different locations, to “one embodiment,” or “an embodiment,” or “an alternative embodiment” in different places in this disclosure do not necessarily refer to the same embodiment. In addition, certain features, structures, or characteristics of one or more embodiments of the present disclosure may be suitably combined.

Furthermore, unless expressly stated in the claims, the order of the processing elements and sequences, the use of numerical letters, or the use of other names as described in this disclosure are not intended to qualify the order of the processes and methods of this disclosure. While some embodiments of the invention that are currently considered useful are discussed in the foregoing disclosure by way of various examples, it should be appreciated that such details serve only illustrative purposes, and that additional claims are not limited to the disclosed embodiments!, rather, the claims are intended to cover all amendments and equivalent combinations that are consistent with the substance and scope of the embodiments of this disclosure. For example, although the implementation of various components described above may be embodied in a hardware device, it may also be implemented as a software only solution, e.g., an installation on an existing server or mobile device.

Similarly, it should be noted that in order to simplify the presentation of the disclosure of this disclosure, and thereby aid in the understanding of one or more embodiments of the invention, the foregoing descriptions of embodiments of the disclosure sometimes group multiple features together in a single embodiment, accompanying drawings, or in a description thereof description thereof. However, this method of disclosure does not imply that more features are required for the objects of the present disclosure than are mentioned in the claims. Rather, claimed subject matter may lie in less than all features of a single foregoing disclosed embodiment.

Some embodiments use numbers to describe the number of components, attributes, and it should be understood that such numbers used in the description of an embodiment are modified in some examples by the modifiers “about,” “approximately,” or “substantially,” “approximately,” or “generally” is used in some examples. Unless otherwise noted, the terms “about,” “approximate,” or “approximately” indicates that a ±20% variation in the stated number is allowed. Correspondingly, in some embodiments, the numerical parameters used in the disclosure and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of this disclosure are approximations, in specific embodiments such values are set to be as precise as practicable.

For each of the patents, patent applications, patent application disclosures, and other materials cited in this disclosure, such as articles, books, disclosure sheets, publications, documents, and the like, are hereby incorporated by reference in their entirety into this disclosure. Application history documents that are inconsistent with or conflict with the contents of this disclosure are excluded, as are documents (currently or hereafter appended to this disclosure) that limit the broadest scope of the claims of this disclosure. It should be noted that in the event of any inconsistency or conflict between the descriptions, definitions, and/or use of terms in the materials appended to this disclosure and those set forth herein, the descriptions, definitions, and/or use of terms in this disclosure shall control. use shall prevail.

Finally, it should be understood that the embodiments described in this disclosure are only used to illustrate the principles of the embodiments of this disclosure. Other deformations may also fall within the scope of this disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A method for magnetic resonance imaging (MRI) implemented on a device including at least one processor and at least one storage device, the method comprising:
applying a gradient echo sequence to an object and performing a dummy scan on the object until a steady-state of the gradient echo sequence is reached;
continuing the dummy scan and maintaining the steady-state of the gradient echo sequence; and
in response to detecting a trigger signal while the gradient echo sequence is in the steady-state, acquiring a gradient echo magnetic resonance signal of the object.

2. The method of claim 1, wherein in response to receiving the trigger signal, the acquiring the gradient echo magnetic resonance signal of the object comprises:
in response to receiving the trigger signal, performing the dummy scan on the object during a trigger delay phase corresponding to the trigger signal;
during a steady-state acquisition phase after the trigger delay phase ends, acquiring the gradient echo magnetic resonance signal of the object.

3. The method of claim 2, wherein the method further comprises:
determining, based on physiological movement features of the object during the trigger delay phase, a number of data acquisitions within the steady-state acquisition phase.

4. The method of claim 1, wherein after acquiring the gradient echo magnetic resonance signal of the object, the method further comprises:
performing a next dummy scan on the object until a next trigger signal is received.

5. The method of claim 4, wherein the performing the next dummy scan of the object until the next trigger signal is received comprises:
in response to determining that a scanning feature of a current phase satisfies a first preset condition, triggering a stop instruction, the stop instruction being configured to pause the next dummy scan.

6. The method of claim 4, wherein the performing the next dummy scan of the object until the next trigger signal is received comprises:
determining, based on the scanning feature of the current phase, a scanning feature of a next phase;
in response to determining that the scanning feature of the next phase satisfies a first preset condition, triggering the stop instruction, the stop instruction being configured to pause the next dummy scan.

7. The method of claim 6, wherein the determining, based on the scanning feature of the current phase, the scanning feature of the next phase, comprises:
determining, by processing the scanning feature of the current phase based on a scanning feature prediction model, the scanning feature of the next phase.

8. The method of claim 5, wherein the method further comprises:
in response to determining that a scanning feature of a last phase does not satisfy the first preset condition and/or in response to determining that a stopping time duration satisfies a second preset condition, triggering a start scanning instruction, the stopping time duration being a length of time from a stopping time point of the dummy scan of the last phase to a current time point, and the start scanning instruction being configured to start the dummy scan.

9. The method of claim 1, after acquiring the gradient echo magnetic resonance signal of the object, the method further comprises:
performing a next dummy scan on the object after a first preset length of time from a time point that acquiring the gradient echo magnetic resonance signal until receiving a next trigger signal.

10. The method of claim 9, wherein the first preset length of time is determined by:
determining, based on a historical trigger interval and a triggering time point of a previous trigger signal, the first preset length of time, the historical trigger interval being an interval length between two adjacent trigger signals.

11. The method of claim 1, wherein the performing the dummy scan on the object comprises:
applying a first radio frequency pulse to the object and simultaneously applying a first selective slice gradient field to the object without acquiring signals.

12. The method of claim 11, wherein the performing the dummy scan on the object further comprises:
applying a first dephasing gradient field to the object after applying the first selective slice gradient field to the object.

13. The method of claim 1, wherein in response to receiving the trigger signal, the acquiring the gradient echo magnetic resonance signal of the object comprises:
applying a second radio frequency pulse to the object and simultaneously applying a second selective slice gradient field to the object;
applying a phase-encoded gradient field and a frequency-encoded gradient field to the object after a second preset length of time to acquire the gradient echo magnetic resonance signal of the object.

14. The method of claim 13, wherein the method further comprises:
applying a second dephasing gradient field to the object before applying the frequency-encoded gradient field to the object;
applying a third dephasing gradient field to the object after applying the frequency-encoded gradient field to the object.

15. The method of claim 1, wherein the method further comprises:
reconstructing, based on the gradient echo magnetic resonance signal, a magnetic resonance image of the object.

16. A system for magnetic resonance imaging(MRI), wherein the system comprises at least one processor; the at least one processor is configured to cause the system to perform operations including:
applying a gradient echo sequence to an object and performing a dummy scan on the object until a steady-state of the gradient echo sequence is reached;
continuing the dummy scan and maintaining the steady-state of the gradient echo sequence; and
in response to detecting a trigger signal while the gradient echo sequence is in the steady-state, acquiring a gradient echo magnetic resonance signal of the object.

17. The system of claim 16, wherein the at least one processor is further configured to cause the system to perform operations including:
in response to receiving the trigger signal, performing the dummy scan of the object during a trigger delay phase corresponding to the trigger signal; during the steady-state acquisition phase after the trigger delay phase ends, acquiring the gradient echo magnetic resonance signal of the object.

18. The system of claim 16, wherein the at least one processor is further configured to cause the system to perform operations including:
reconstructing, based on the gradient echo magnetic resonance signal, a magnetic resonance image of the object.

19. A non-transitory computer readable medium storing instructions, the instructions, when executed by at least one processor, causing the at least one processor to implement a method comprising:
applying a gradient echo sequence to an object and performing a dummy scan on the object until a steady-state of the gradient echo sequence is reached;
continuing the dummy scan and maintaining the steady-state of the gradient echo sequence;
in response to detecting a trigger signal while the gradient echo sequence is in the steady-state, acquiring a gradient echo magnetic resonance signal of the object.

20. A system for magnetic resonance imaging(MRI), wherein the system is configured to communicate with a terminal device of a user, in response to receiving configuration information from the terminal device of the user, the system performs, based on the configuration information, operations including:
applying a gradient echo sequence to an object and performing a dummy scan on the object until a steady-state of the gradient echo sequence is reached;
continuing the dummy scan and maintaining the steady-state of the gradient echo sequence; and
in response to detecting a trigger signal while the gradient echo sequence is in the steady-state, acquiring a gradient echo magnetic resonance signal of the object.
